(19) United States
(12) Patent Application Publication
(10) Pub. No.: US 2021/0055284 A1
(43) Pub. Date: Feb. 25, 2021

(54) MICROCHIP IMMUNOASSAY DEVICE HAVING PRECISE INCUBATION TIME CONTROL AND SIGNAL SCALING AND RELATED METHODS

(71) Applicant: Zding Tech LLC, Pittsford, NY (US)

(72) Inventor: Zhong Ding, Pittsford, NY (US)

(21) Appl. No.: 16/997,369

(22) Filed: Aug. 19, 2020

Related U.S. Application Data
(60) Provisional application No. 62/889,241, filed on Aug. 20, 2019.

(51) Int. Cl. G01N 33/53; G01N 33/558; B01L 3/00

(52) U.S. Cl. CPC G01N 33/5302; G01N 33/558; B01L 2300/0816; B01L 3/502776; B01L 2200/10; B01L 3/5023

(57) ABSTRACT

A lateral flow (immunoassay) device retains at least one assay chip inside a solid frame. Each assay chip includes a sample zone, a conjugate zone, capture zone(s) and a waste zone. The sample and waste zones each include a hydrophilic pad sitting above the chip. The frame is hydrophobic, having a fluid metering window and optional air vent in the top and a scan window provided at the bottom. A sample with analyte is dispensed with the volume of sample such that fluid flow stops when solution with dissolved conjugate reaches a designated location in the chip, and at least a portion of the waste zone is still dry. A second fluid is subsequently added to wash off unbound conjugate in the capture zone after the device with first fluid has been incubated for an assay specified time period. During incubation and after wash, multiple optical scans are performed to obtain conjugate signal profiles along the chip. The features of the signal are used to define an assay signal read location. The bound conjugate signal after wash is then scaled by the total conjugate signal prior to wash at the read area. This scaled signal is defined as assay response. A single device can perform single test for one sample as well as multiple tests for a single or multiple samples.

### CROSS REFERENCE TO RELATED APPLICATION

This application claims priority to U.S. Patent Application Ser. No. 62/889,241, entitled: A Microchip Immunoassay Device Having Precise Incubation Time Control and Signal Scaling and Related Methods, filed Aug. 20, 2019, under relevant portions of 35 U.S.C. § 119, the entire contents of which are incorporated by reference.

### TECHNICAL FIELD

This application is directed generally to the field of diagnostic medicine, and more specifically to an improved lateral flow device for immunoassays and methods relating to the improved lateral flow device.

### BACKGROUND

Diagnostic assays are used for detecting analyte concentration in a clinical sample (such as blood, serum, plasma, and urine) to assist the diagnosis, treatment and management of many diseases.

To satisfy the need for a fast, accurate, low cost, and easy to use test, many different types of diagnostic assay devices have been developed over the years.

A lateral flow assay device is one of the common technologies for diagnostic assay applications. These devices typically include a zone for receiving a liquid sample, a reagent zone to supply labeled conjugate (soluble and movable), a reaction zone for assay specific conjugate capture and detection, and a waste collection zone for capillary flow and waste fluid storage.

In a typical lateral assay process, liquid sample is applied to the sample zone. Driven by capillary pressure, sample flows downstream to dissolve the deposited conjugate. The dissolved conjugate or conjugate-analyte complex binds to capture antibodies deposited in the capture zone as fluid sample flows through the capture zone to the waste zone. The unbound conjugate is washed off from the capture zone by the flowing sample or an added wash buffer after all the deposited conjugate is dissolved. Flow will stop when the waste zone is saturated by the fluid and a signal detection for the captured conjugate will be performed in the capture zone to determine the analyte concentration.

One type of lateral flow device typically employs a hydrophilic porous material, e.g., nitrocellulose, defining a path for fluid flow driven by capillary pressure. Examples of these devices are shown in U.S. Pat. Nos. 5,559,041, 5,714,389, 5,120,643, and 6,228,660.

Another type of lateral flow assay device employs hydrophilic non-porous material, with capillary structure such as flow channels or a plurality of upwardly extending projections configured to induce capillary flow. Examples of such devices are disclosed in U.S. Pat. No. 8,025,854 B2, WO 2003/103835, WO 2005/089082, WO 2005/118139 and WO 2006/137785.

The sample-receiving zone of the lateral flow devices may be capable of separating blood cells if whole blood is used as the sample. Examples of separating materials are typically fibrous materials (such as cellulose, wool, glass fiber, asbestos, polymers, or mixtures of the same).

An instrument such as that disclosed in U.S. Patent Application Publication No. 2006/0289787 A1, U.S. Patent Application Publication No. 2007/0231883 A1, and U.S. Pat. Nos. 7,416,700 and 6,139,800, all incorporated by reference in their entireties herein, is configured to detect the bound conjugated material in the detection zone. Common labels include fluorescent dyes that can be detected by instruments which excite the fluorescent dyes and incorporate a detector capable of detecting the resulting fluorescence.

In the foregoing devices and in the conduction of assays, the resulting level of signal in the detection zone is read using a suitable detection instrument after the conjugate material has all been dissolved and sample and unbound conjugate material and wash fluid added to a reagent zone of the device has reached and subsequently filled the wicking zone of the device.

A major issue with the above lateral flow assay techniques is that of fluid flow rate inconsistency and conjugate dissolution inconsistency due to its sensitivity to device manufacture imprecision (the material surface property and the structure geometry). Fluid property (e.g., viscosity) differences also lead to flow rate differences among samples. Fluid flow may be extremely slow or even stop due to device manufacture issues or sample fluid property issues (e.g., very high viscosity).

This flow inconsistency among tests leads to variations in conjugate dissolution time as well as conjugate concentration in the capture zone, and therefore also affecting the binding reaction time and the rate between conjugate and the capture antibody, leading to variation of the bound conjugate among the tests with poor assay precision.

A second issue is that variations in conjugate morphology or conjugate pad location from manufacture also leads to variations in conjugate dissolution time and conjugate concentration profile in the capture zone, contributing to assay imprecision.

A third issue is that for a non-porous lateral flow device, the capture antibody is typically deposited on the solid surface of the capture zone, which is prone to so-called “coffee ring” effects (i.e., multi-layers of capture antibody piled up instead of a mono-layer, especially near the boundary of the deposited spot). These effects will not only affect fluid flow, but also lead to lose of signal due to wash off of the bound conjugate and capture antibody complex during flow.

A fourth issue for the above stated lateral flow assay technique is that the device size is large (typically in inches) due to the requirement of sufficiently long flow time. The large consumable size adds cost and makes the devices harder to process, particularly for use in high throughput automatic instruments due to the device's bulky size.

A fifth issue is the lack of flexibility in assay incubation time. For example, some assays may require a short incubation time (e.g., 5 minutes) while others may need a long incubation time (e.g., 20 minutes). It is unlikely that one device with the same geometry and material can satisfy both short and long incubation time requirements. To satisfy the different requirements, multiple designs and/or materials have to be used, which increase the cost and manufacture complexity.

Due to poor assay precision, high unit cost, and bulky device sizes, currently known lateral flow techniques are woefully inadequate for high throughput instrument applications.

Accordingly, there is a perceived need for a device in which assay incubation time is precise with little effects from fluid flow inconsistency. There is also a need for a device that can satisfy the requirement of a wide range of assay specific reaction times (e.g., from 2 to 40 minutes) among different assays without changing device material or geometry. It is desirable that assay incubation time (the reaction time) is independent of variations in fluid properties, construction materials, and device manufacture.

There is also a need in the field to reduce the assay sensitivity to conjugate concentration variation in the capture zone associated with device manufacture (conjugate deposition or pad location), material (pore distribution and geometry, surface properties, etc.), or fluid flow.

There is a further prevailing need to reduce the size of the device for easier consumable handling, for use in conjunction with more compact testing instruments, and for lowering overall material and manufacturing costs.

It is also desirable that the capture zone of the lateral flow device has no “coffee ring” effects resulting from manufacture.

It is also important to be able to detect flow irregularities in order to ensure assay quality for the lateral flow assays.

### BRIEF DESCRIPTION

According to at least one aspect, there is provided a lateral flow device for conducting immunoassays including a chip disposed inside a solid frame. The frame surface is typically hydrophobic. An opening in top of the frame cover exposes a sample pad, which is typically a hydrophilic porous film structure (e.g., a fiber glass filter). The frame further includes a scan window to allow for optical scanning of the chip. In at least one version, the scan window is formed in the bottom of the frame cover, although according to at least one embodiment the scan window can alternatively be disposed at the top of the frame cover. The chip is a hydrophilic porous strip defined by multiple zones. One end of the chip is in direct contact with the sample pad, which receives fluid to the chip via the opening formed in the top of the frame. More specifically, the sample pad and the portion of the chip in direct contact with the sample pad defines a sample zone. Downstream relative to the sample zone in the chip is a conjugate zone in which labeled conjugates are deposited. Downstream to the conjugate zone is a capture zone preferably having capture antibody (AB) coated beads trapped inside the porous or capillary structure of the chip. Downstream relative to the capture zone is a waste zone, which includes an absorption pad as well as the portion of the chip that the absorption pad contacts directly.

To perform an assay test in accordance with the present invention, the lateral flow device requires two (2) separate fluid dispenses at an assay specific time interval in order to precisely control the assay incubation time. The first dispensed fluid is the patient sample (or any fluid containing an analyte of interest) that requires the detection of the presence of specified analytes and/or their concentrations in the sample. The second dispensed fluid, which can be a wash buffer or the original sample fluid, is designed to remove the unbound conjugate from the capture zone.

After the dispense of the first fluid to the sample zone, the first fluid flows under porous or capillary action to the conjugate zone and dissolves the labeled conjugate. The sample with the dissolved conjugate continues flowing downstream to the capture zone and then stops. The sample volume of the first fluid is determined in a way such that sample fluid flow stops after the peak of the dissolved conjugate reaches the desired location (e.g., inside or after the capture zone), and while the waste zone is still at least partially dry. Bulk fluid flow will stop after the sample fluid above the sample zone is depleted due to zero gradient in capillary pressure, although microscopic fluid flow from larger pores to the smaller pores continue.

The device with the sample dissolved conjugate in the capture zone is then incubated for an assay specific time period (e.g., 5 minutes) at a specified temperature (e.g., 37 degrees C.) without bulk fluid flow. Fluid flow resumes with the second fluid (e.g., the wash fluid) added to the sample zone (or another upstream location), washing off unbound conjugate from the capture zone. Fluid flow will stop again when the waste zone is saturated by fluid or the wash fluid above the sample zone (or the upstream dispense location) is depleted completely.

Following the first sample fluid dispense, the sensor system of the detection instrument (e.g., the CCD camera, the LED and photodiode pair, or the other optical system) scans along the chip covering at least part of the conjugate zone, the entire capture zone, and at least part of the waste zone at specified excitation wavelengths in order to obtain the conjugate signal (e.g., the emission light at another wavelength) profiles corresponding to the conjugate concentration as function of both time and location along the chip. Preferably, multiple scans are performed with at least two (2) critically required scans. A first scan is performed immediately before wash fluid addition to obtain the total conjugate (both free and bound) signal distribution. The second scan is performed after wash completion in order to obtain the bound conjugate signal in the capture zone.

The time interval between the two fluid dispenses is assay specific and controllable by design. For example, a fluid metering pipette or other dispensing means dispenses the sample fluid at a starting time to the sample zone of the lateral flow device in order to initiate the immunoassay reaction. The dispensing means (e.g., pipette) then waits for an assay specific incubation time before dispensing the second (e.g., wash) fluid. The flow time after sample dispense is short (e.g., typically less than 5 seconds) due to a very short flow distance (e.g., less than 16 mm), a low contact angle, and a high permeability of the porous structure (e.g., fiber glass) or capillary structure. The variation in this short flow time contributes little to the total assay time (typically a few minutes or more).

The scanned conjugate concentration profiles can be used for quality detection, read location detection, assay signal scaling, as well as assay response calculation as described below:
(1) The concentration profiles can be used for determining if the lateral flow device is performing normally. An erratic conjugate concentration profile (e.g., the peak of the profile being outside a predetermined allowable range, presence of spikes in the read area, etc.) may indicate errors in fluid flow, device manufacture, or other issues. An alert can be created in which the detected signal may not be used for analyte concentration prediction.
(2) Determining the read location based on the conjugate profiles prior to and/or after wash for assay signal and assay response calculation. The most significant feature in the total conjugate prior to wash is the signal peak.
(3) Obtaining a normalization value (peak value, mean value, medium value, or total value) of the total conjugate signal (both free and bound) in the read area prior to wash addition. Here the read area is a region centered at the read location determined by the conjugate profiles or specific locations known by design. The read area is typically rectangular in shape.
(4) Scaling the scanned signal after wash completion with the normalization value.
(5) Calculating the assay response with the scaled signal in the read area.

Assay concentration corresponding to the assay response is calculated with the established dose response curve.

Accordingly, various configurations or embodiment for the herein described immunoassay (lateral flow) device can be provided for different applications. For example:
(1) According to at least one embodiment, a basic construction of the lateral flow device includes a single chip within a frame including a single capture zone and one labeled conjugate for one assay test. This device design is intended for use with one sample with one assay test.
(2) In accordance with another embodiment, the lateral flow device can include multiple identical chips inside a frame with all the chips being isolated fluidically. Each chip according to this device design utilizes a single capture zone and one labeled conjugate in the conjugate zone for one assay test each. This device configuration is used for the same assay test across multiple samples (e.g., multiple patients or same patients with multiple reps).
(3) In accordance with at least one other embodiment, the lateral flow device can include multiple chips inside a frame with one capture zone and one labeled conjugate dedicated for each chip. According to this device configuration, each chip performs a unique assay test in which there are no fluidic communications among the multiple chips. This device design is configured for multiple tests involving one sample (e.g., panel tests for a sample), or for different assay tests of different patient samples.
(4) In accordance with yet another version, the lateral flow device can include a single chip inside a frame with multiple capture zones sequentially arranged along the chip for multiple assay tests. Multiple labeled conjugates corresponding to multiple analytes from one sample are deposited in a single conjugate zone. This device configuration enables multiple tests to be conducted for one patient sample.
(5) In accordance with one other embodiment, the lateral flow device can include multiple chips inside a frame with multiple tests in each chip. Each chip is the replicate of the others, performing the same multi-tests with multiple capture zones that are sequentially arranged along the chip for multiple assay tests. The labeled conjugates corresponding to the multiple analytes from one sample are deposited in one conjugate zone in each chip. In this device design, there are no fluidic communications among the chips in which this lateral flow device configuration is intended for multiple samples with the same multi tests.
(6) In accordance with yet another variation, the lateral flow device can include multiple chips inside a frame with multiple tests in each chip. According to this configuration, each test across all the chips is unique in which each chip has multiple capture zones sequentially arranged along the chip for multiple assay tests. The labeled conjugates corresponding to the multiple analytes from one sample are deposited in one conjugate zone in each chip. There are no fluidic communications among the chips in which this particular device configuration can be used for large panel tests of a single sample, or multiple samples with different tests.

For multiple chips in a device, each chip can be scanned separately either by multiple optical sensors, or by a single optical sensor such as a CCD camera.

For the present invention, a first unique feature as compared to other lateral flow methods is that the sample volume is predefined such that sample fluid flow will stop after the sample dissolved conjugate reaches the capture zone for incubation. More specifically, the sample volume is determined in a way such that bulk sample fluid flow stops after the peak of the dissolved conjugate reaches the desired location (e.g., inside the capture zone or just downstream to the capture zone) while the waste zone is still at least partially dry. The device with the sample dissolved conjugate in the capture zone is then incubated for an assay specific time period (e.g., 5 minutes) at a specified temperature (e.g., 37 degrees C.) without bulk fluid flow although microscopic flow from larger pores to smaller pores continue.

A second unique feature of this invention is the addition of a second fluid after the sample is incubated in the chip for an assay specific incubation time. Fluid flow resumes with the second fluid (either the same sample or a wash buffer) addition to the sample zone (or another location upstream), washing off unbound conjugate from the capture zone. Fluid flow will stop again until the waste zone is saturated or the second dispensed fluid above the sample zone is depleted completely.

A third unique feature of the invention is the incorporation of trapped beads to create a pore size gradient across the thickness of the conjugate zone before conjugate deposition. This gradient makes conjugate fluid flow slower at the layers having more beads, and faster at the layers having little or no beads present. The flow velocity differences across the thickness of the conjugate zone leads to dissolved conjugate spreading wider with a smaller gradient along the fluid flow direction to cover the capture zone.

A fourth unique feature of the invention is the creation of a very wide (e.g., 8 mm) capture zone along the chip fluid flow direction, while maintaining a relatively smaller read area (e.g., 1.5 mm) and searching for the read location using the signal peak (either total conjugate prior to wash or bound conjugate peak after wash). The read location can be the peak, or a fixed distance from the peak. By finding the read location for a more consistent conjugate concentration instead of using a predefined fixed read location, the device is more tolerable to sample volume or manufacture variations.

A fifth unique feature of the invention is in scanning the chip multiple times along the chip's longitudinal (i.e., flow) direction after sample addition until the completion of the test, covering at least part of the conjugate zone, the entire capture zone, and at least part of waste zone at specified wavelengths to obtain conjugate signal profiles corresponding to the conjugate concentration as a function of both time and location along the chip. The scan performed immediately before the addition of the second (wash) fluid is required to obtain the total conjugate signal, which will be used to obtain a scaling factor (the normalization value) in the read area at the read location of the capture zone. The scan after wash completion is required to obtain the bound conjugate signal in the read area at the read location of the capture zone. The conjugate signal profiles can be used to determine the read location, to obtain the normalization value (the scaling factor) at the read area, to scale the signal, to obtain assay response, and to detect errors.

A sixth unique feature of this invention is in defining the assay response as the scaled signal (the signal after wash is scaled by the signal obtained prior to wash at each read area). This scaling significantly improves assay precision, making the device less sensitive to fluid volume and other variations.

A seventh unique feature of this invention is in providing lateral flow device designs that are configured for multi-tests and/or multi-patient tests capabilities. All of the chips in a multiple chip device design are separated without liquid communications among each other. Each chip has its own fluid metering window. The nubs or other spacer structure within the device interior maintains the chip in a preferred position while minimizing the chance for wicking flow between the chip and the frame. The sample pad is sealed against the top cover. The absorption pad as part of waste zone is optional. The vent hole at the cover is also optional, since the device is not air tight between the chip and the scan window.

A number of further advantages can be realized using the herein described device and related method(s), which minimally include:

Precision and sensitivity
Precise assay specific immuno-assay reaction time
Dynamic read location determination based on dissolved conjugate profiles
Minimized effects of conjugate variation with signal scaling technique
Larger surface area with shorter diffusion distance
Minimized non-specific binding to capture Ab bead surface with blocking
No coffee-ring effects in capture area
   Low cost
No liquid reagents with tiny dry device
Shared chip manufacture format for various configurations
Batch production process
Simple inventory management (transportation, storage, and waste handling)

### Multiplexing

Multi-patients with same test per device
Multi-tests per device for a patient

These and other features and advantages will be apparent from the following Detailed Description, which should be read in conjunction with the accompanying drawings.

### BRIEF DESCRIPTION OF THE DRAWINGS

FIGS. 1(a) and 1(b) depicts side and top views, respectively, of a lateral flow device made in accordance with aspects of the present invention;

FIGS. 2(a)-2(e) depicts the working principal of the lateral flow device sequentially from the addition of first fluid (sample), sample flow with conjugate, flow stop with incubate, second fluid addition, and optical scan;

FIG. 3 is an exploded view of a lateral flow device in accordance with aspects of the present invention;

FIGS. 4(a) and 4(b) are top and bottom perspective views of an exemplary lateral flow device having the configuration of the device of FIG. 3;

FIGS. 5(a) and 5(b) present a side elevational and top view of an exemplary lateral flow device along with typical geometric dimensions;

FIG. 6 is a side elevational view of a lateral flow device in accordance with other aspects of the invention, in which the conjugate zone is partially beneath the sample pad;

FIG. 7 is an enlarged schematic side view of a portion of an exemplary lateral flow device in accordance with aspects of the invention in which a plurality of beads are trapped at one side of the chip porous medium;

FIG. 8 depicts a profile of conjugate signal versus location, as taken from an exemplary lateral flow device;

FIG. 9 illustrates an enlarged schematic side view of a portion of an exemplary lateral flow device in accordance with aspects of the invention and more specifically an exemplary capture zone;

FIGS. 10(a)-10(c) depict conjugate profiles of exemplary lateral flow devices having a read location and read area for short or long capture zones, respectively, of an exemplary device;

FIG. 11 illustrates a total conjugate concentration profile prior to wash and the bound conjugate after wash for a short capture zone of an exemplary lateral flow device in accordance with aspects of the invention;

FIG. 12 depicts total conjugate concentration profile prior to wash and the bound conjugate after wash for a long capture zone in accordance with aspects of the inventive lateral flow device;

FIG. 13 depicts various conjugate profiles illustrating peak location shifts occurring for different sample volumes;

FIG. 14 illustrates conjugate signal profiles for an exemplary lateral flow device having multi-capture zones;

FIG. 15 depicts exemplary competitive assay reactions;

FIG. 16 depicts a mathematical model relating to the kinetics of a competitive assay;

FIGS. 17(a) and 17(b) are graphical profiles illustrating the kinetics between a faster competitive assay and a slower competitive assay and more specifically denoting variations in concentration over a period of reaction time;

FIGS. 18(a)-18(d) depict graphical profiles indicative of dose response for faster competitive assays and predicated concentration bias for faster competitive assays in accordance with aspects of the invention;

FIGS. 19(a)-19(d) illustrate graphical profiles indicative of dose response for slower competitive assays and predicated concentration bias for slower competitive assays in accordance with aspects of the invention;

FIG. 20 illustrates a top perspective exploded assembly view of another exemplary lateral flow device made in accordance with aspects of the invention;

FIGS. 21(a) and 21(b) depicts top and bottom perspective views of the exemplary lateral flow device of FIG. 21;

FIG. 22 illustrates a top perspective exploded view of an exemplary lateral flow device made in accordance with other aspects of the invention;

FIGS. 23(a) and 23(b) are top and bottom perspective views of the lateral flow device of FIG. 22;

FIG. 24 illustrates an exploded top perspective assembly view of an exemplary device made in accordance with another version;

FIGS. 25(a) and 25(b) depict top perspective and bottom perspective views of the lateral flow device according to FIG. 24;

FIG. 26 illustrates a top perspective exploded assembly view of an exemplary lateral flow device in accordance with other aspects of the invention;

FIGS. 27(a) and 27(b) depict respective top perspective and bottom perspective views of the lateral flow device of FIG. 26;

FIG. 28 illustrates a top perspective exploded assembly view of another exemplary lateral flow device in accordance with aspects of the invention;

FIGS. 29(a) and 29(b) depict respective top perspective and bottom perspective views of the lateral flow device of FIG. 28;

FIG. 30 illustrates a top exploded perspective view of an exemplary lateral flow device in accordance with other aspects of the invention;

FIG. 31 illustrates a top perspective view of the lateral flow device of FIG. 30; and

FIG. 32 depicts experimental data depicting conjugate signal prior to wash and during incubation, and after wash.

### DETAILED DESCRIPTION

The following description relates to various embodiments of lateral flow devices, as well as related methods of use. It will be readily apparent from the following discussion that variations and modifications can be made by those of sufficient skill to the device designs that are discussed. In addition, several terms are used throughout to provide a suitable frame of reference in regard to the accompanying drawings. These terms are not intended to limit intended scope of the inventive concepts, except where so specifically indicated.

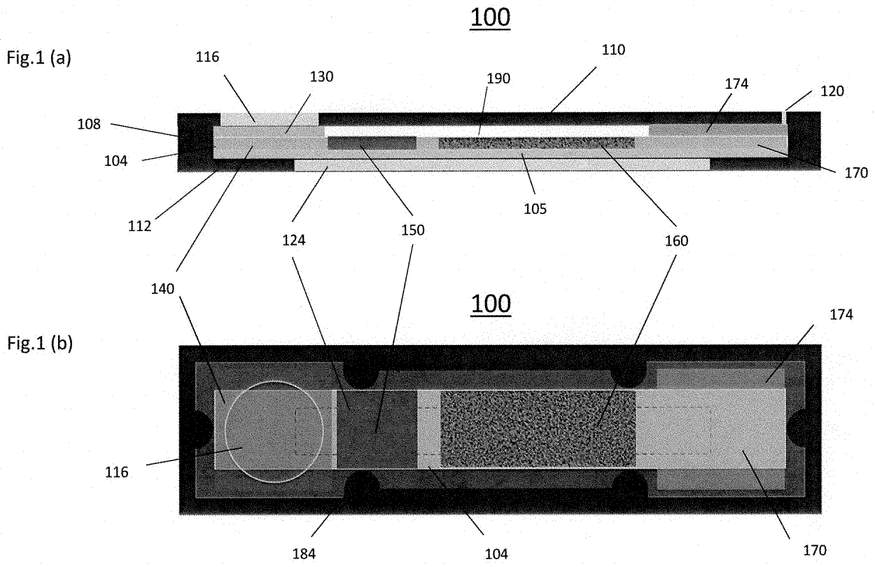

With reference to FIGS. 1(a) and 1(b), there is shown an immunoassay device (also referred to throughout this discussion as a “lateral flow device”) 100. The immunoassay device 100 includes an assay chip 104 defined by a hydrophilic porous structure, which is disposed within the hollow interior of a solid frame 108, the frame 108 including a top cover 110 and a bottom cover 112. A fluid metering window 116, as well as an air vent 120 are formed in the top cover 110, and a scan window 124 is similarly formed in the bottom cover 112 according to this specific embodiment. The assay chip 104 is typically an elongated strip, constructed using either porous material (e.g., glass fibers) with or without a solid support layer (e.g., surface modified polystyrene, polycarbonate, polyolefin, or polyethylene terephthalate) with capillary channels, or multilayers of solid beads (e.g., surface modified polystyrene beads) on a plastic support, or a molded capillary structure. Fluid flow though the assay chip 104 is driven by surface energy (i.e., capillary pressure gradient). A hydrophilic porous sample pad 130 is sealed to the top cover 110 at the fluid metering window 116 and fluidically connected with the assay chip 104 at the left end, as shown in FIG. 1(a). The hydrophilic porous sample pad 130, which according to this embodiment is sealed beneath the top cover 110, fluidically connects with the assay chip 104 at the depicted left end of the chip 104. The vent 120 provided in the top cover 110 of the solid frame 108 of the lateral flow device 100 allows air flowing out from inside the solid frame 110. Since the herein described lateral flow device 100 is not air tight (e.g., between the assay chip 104 and the scan window 124), the air vent 120 can be provided as an optional feature.

The herein described assay chip 104 is defined by a plurality of adjacent zones. First, a sample zone 140 of the herein described lateral flow device 100 is defined by the structure including the porous sample pad 130 and the portion of the assay chip 104 that is fluidically connected to the sample pad 130. Adjacent to the sample zone 140 and downstream therefrom according to this embodiment is a conjugate zone 150 having labeled conjugates 154 (a monoclonal or polyclonal antibody linked to a label). The labels can include fluorescent molecules, such as fluorescein isothiocyanate (FITC), tetramethylrhodamine isothiocyanate (TRITC), cyanine (Cy3), or phycoerythrin (R-PE). Other suitable labels can include enzymes, such as horseradish peroxidase and alkaline phosphatase for colorimetric detection.

Adjacent and downstream relative to the conjugate zone 150 is a capture zone 160 in which a plurality of micro-beads with coated antibodies on surfaces are trapped inside the porous structure (or capillary structure) of a portion of the elongated assay chip 104. Finally, a waste zone 170 (also referred to throughout this discussion as the “absorption zone”) is defined by the structure including an absorption pad 174 and the portion of the assay chip 104 that the absorption pad connecting underneath. The waste zone 170 is downstream from the capture zone 160 of the herein described lateral flow device 100 and configured and sized to absorb fluid until fully saturated. The waste zone 170 may adequately function in accordance with or without the absorption pad 174. The purpose of the absorption pad 174 is to increase waste fluid absorption capacity in a smaller chip dimension. An air gap 190 between the top cover 110 and the assay chip 104, as shown in FIG. 1(a), is optional. The foregoing relates to an exemplary device design. As discussed, the number of assay chips 104, as well as the number of sample and absorption pads 130, 174 can be suitably varied.

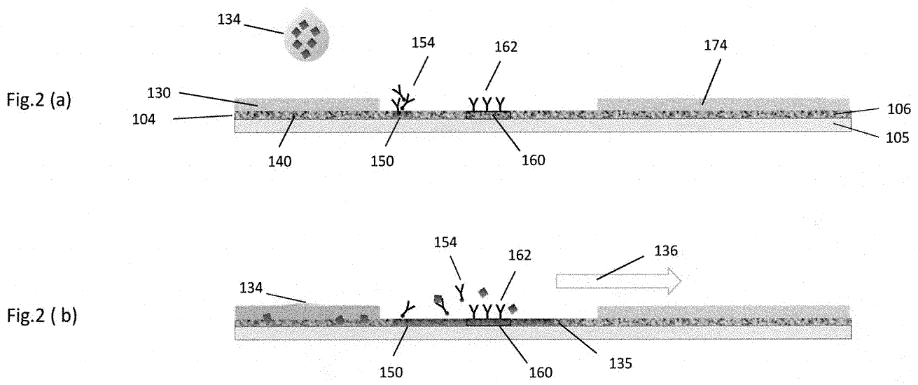

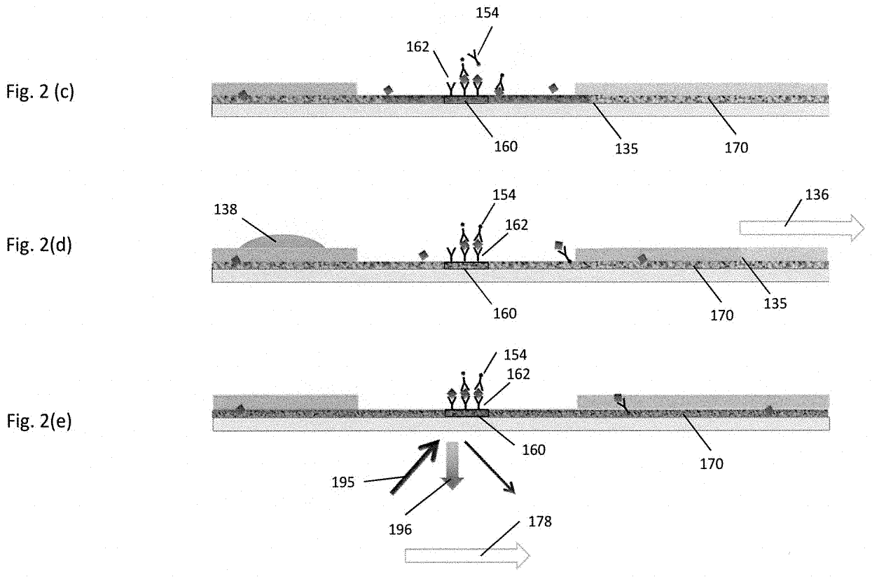

FIGS. 2(a)-2(e) illustrates the working principal of the lateral flow device 100 according to FIGS. 1(a) and 1(b), as well as other device variants, as described herein. The frame including the top and bottom covers is not shown in FIGS. 2(a)-2(e) for reasons of clarity. Illustrated is a sandwich assay format in which the assay chip 104 of the lateral flow device 100 is connected with the sample and absorption (waste) pads 130 and 174 by direct contact. The assay chip 104 is defined by a base or support layer 105 and a porous layer 106 that enables fluid transport laterally. Preferably, the base layer 105 is transparent.

First and with reference to FIG. 2(a), a quantity of sample fluid 134 containing at least one analyte of interest is dispensed using a pipette or other suitable dispensing means (not shown) into the sample pad 130 via the fluid metering window 116 at the sample zone 140 of the device 100.

Next and as shown in FIG. 2(b), the dispensed sample 134 flows from the sample zone 140 (the sample pad 130 and the portion of the assay chip 104 in direct contact with the sample pad 130) per the flow direction shown in arrow 136 to the conjugate zone 150, dissolving the dry labeled conjugate 154, and moving the solution containing dissolved conjugate along with the sample fluid to the capture zone 160 with an observable moving wet-dry interface 135 as the fluid flows along the chip 104 driven by a capillary pressure gradient between the interface 135 and the sample fluid 134 above the sample pad 130. Typically, the labeling is fluorophore-based. The capture zone 160 includes a plurality of microbeads having capture antibodies 162 disposed therein. Fluid flow will continue as long as there is sample fluid 134 above the sample pad 130.

Next and as shown in FIG. 2(c), fluid flow stops in the macroscopic scale as sample fluid 134 above the sample pad 130 depletes and sample fluid 134 fully enters the sample pad 130. This fluid stoppage is due to the minimized capillary pressure gradient between the sample pad 130 and the wet-dry interface 135 in the chip 104. In a microscopic scale, fluid will continue to flow from larger pores to smaller pores. The solution with conjugate, analyte, and capture antibodies in the capture zone 160 is incubated for an assay specific time period.

Next and as shown in FIG. 2(d), a quantity of a second (e.g., wash) fluid 138 is added to the sample pad 130 following incubation and fluid flow resumes. The unbound conjugate is removed from the capture zone 160 with fluid flowing toward the waste zone 170. Subsequently and as shown in FIG. 2(e), the bound conjugate in the capture zone 160 is detected optically using a suitable optical scanning instrument (shown schematically as 176) via the scan window 124 in the bottom cover 112 of the lateral flow device 100 and scans along the chip 104 through the conjugate zone 150, capture zone 160, and part of the waste zone 170 in the direction 178. or fluorescence detection (e.g., with a CCD camera or an LED and laser diode pair of a suitable scanning instrument (not shown)), the excitation wavelength (λ₁) 195 is always different from the emission wavelength (λ₂) 196.

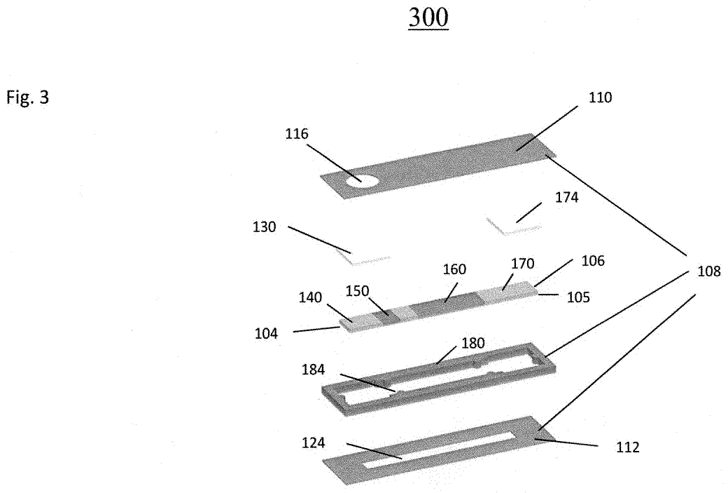

FIG. 3 is an exploded assembly view of the components of a lateral flow device 300 in accordance with an exemplary embodiment. Similar parts are labeled with the same reference numbers for the sake of clarity. The herein described lateral flow device 300 includes a solid frame 108 that includes a top cover 110 and a bottom cover 112. The top cover 110 includes a fluid metering window 116 and an optional vent hole (not shown in FIG. 3, but shown as 120 in FIG. 1). The bottom cover 112 includes a scan window or opening 124 in which the surfaces of the top and bottom cover 110, 112 are typically hydrophobic. The assay chip 104, which as previously discussed is an elongated member, typically has a transparent support layer 105 (a backing layer) glued (or otherwise bound) to a hydrophilic porous layer 106. The sample pad 130 is also fabricated from a hydrophilic porous material (e.g., fiber glass, filter papers, or nitrocellulose membranes) and is sealed against the interior side of the solid top cover 110 either by glue or compression against the cover 110. The sample pad 130 is exposed to ambient air through the fluid metering window 116 at the top cover 110 to receive delivered fluid. The sample pad 130 is in direct contact with the hydrophilic porous layer 106 of the assay chip 104 underneath so that fluid in the sample pad 130 flows to the assay chip 104, and is driven by capillary pressure. The front of the assay chip 104 is connected with the sample pad 130 to form the sample zone 140. The porous structure has soluble labeled conjugates 154, FIG. 2(a), deposited on the assay chip 104 in the conjugate zone 150. Downstream relative to the conjugate zone 150 according to this embodiment are beads 162, FIG. 2(a), with capture antibody trapped in the porous structure of the assay chip 104 in the capture zone 160. The back end of the assay chip 104 as shown is fluidically connected with the absorption (waste) pad 174 to form the waste zone 170. The absorption pad 174 is made from a porous hydrophilic material (e.g., fiber glass, nitrocellulose, etc.) that is designed to absorb liquid.

In use, dispensed fluid flows from the sample pad 130 to the hydrophilic porous layer 106 of the assay chip 104 underneath, then to the conjugate zone 150, and then the capture zone 160. Fluid flow pauses for an assay specific time after sample fluid is depleted above the sample pad 130 with the dissolved conjugate 154 covering the capture zone 160. Fluid flow then continues to the waste zone 170 with the addition of the second (wash) fluid in the sample pad 130. The unbound conjugate in the capture zone 160 is removed by the addition of the wash fluid. According to this device embodiment, the solid frame 180 defined by the top and bottom cover further includes, according to this version, a hydrophobic spacer structure 180 that includes a plurality of nubs 184. The spacer structure 180 offers rigidity and space for the lateral flow device 300 in order to house the assay chip 104. The nubs 184 are disposed so as to maintain the assay chip 104 in position, while minimizing the chance of wicking flow of fluid between the frame 108 and the assay chip 104. Moreover, the frame 108 is configured to hold steady the two pads 130, 174 with the assay chip 104 underneath. Direct physical contact between the sample and absorption pads 130, 174 and the assay chip 104 is ensured by the pressing from the top and bottom cover 110, 112 of the herein described lateral flow device 300.

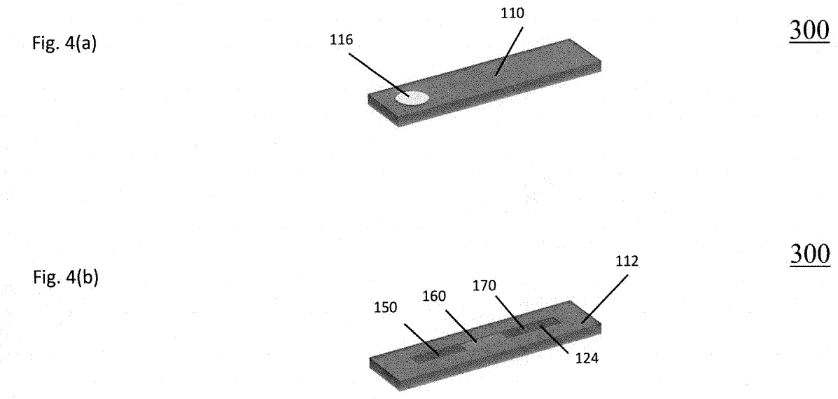

FIGS. 4(a) and 4(b) depict assembled top and bottom perspective views of the lateral flow device 300 shown in FIG. 3. As discussed, the opening 116 formed in the top cover 110 shown is for fluid metering to the sample pad 130. According to this embodiment, the scan window 124 formed in the bottom cover 112 is configured to expose the chip conjugate zone 150, the entire capture zone 160, and at least a portion of the waste zone 170. The scan window 124 according to this version is substantially centered at the center of the width of the assay chip 104, the scan window 124 extending in the longitudinal direction of the bottom cover 112. The foregoing lateral device design 300 includes a single assay chip 104 for performance of a single assay test in which the conjugate and capture zones 150, 160 of this device 300 are visible for scan through the scan window 124 provided in the bottom cover 112. Visibility of the conjugate zone 150 through the scan window 124 is optional for quality monitor purposes only.

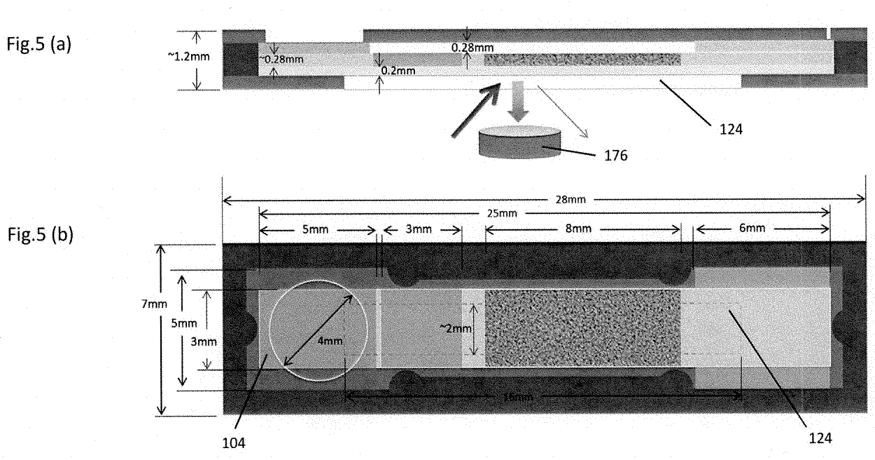

FIGS. 5(a) and 5(b) illustrate an exemplary lateral flow device of the type 300 depicted in FIGS. 3, 4(a) and 4(b), and further including typical dimensions. In this example, the width dimension of the scan window 124 is narrower than the width dimension of the assay chip 104. More specifically and in this specific example, the width of the scan window 124 is 2 mm while the width of the assay chip 104 is 3 mm. The optical sensor 176 is disposed beneath the scan window 124 and configured to longitudinally scan along the scan window 124, as discussed.

The following provides a more detailed description for each zone of the inventive lateral flow device 100, 300.

### The Sample Zone:

The hydrophilic sample zone 140 is either a porous structure or a solid capillary structure. This zone 140 receives sample fluid and transfers the sample towards the conjugate zone 150. The sample zone 140 therefore serves as an interface between a fluid delivery device (e.g., a pipette) and the conjugate zone 150.

The sample pad 130 in the sample zone 140 promotes a more even and controlled distribution of a sample fluid onto the conjugate zone 150 downstream and minimizes the effects of sample fluid deliveries (e.g., variations in dispense location, dispense rate, or interface geometry of the fluid delivery device).

For the sample zone designs described herein, the sample pad 130 (e.g., a fiber glass filter) is sandwiched between the top cover 110 of the solid frame 108 at the fluid metering window 116 and the porous structure (hydrophilic porous layer 106) of the assay chip 104. Dispensed fluid flows from the sample pad 130 to the porous structure of the chip 104 underneath, and then flows downstream to the conjugate zone 150. The fluid metering window 116 is an opening (a round, rectangular, or other geometric shape) in the frame's top cover 110 so that sample and/or wash fluid can be delivered to the sample pad 130. The sample pad 130 is either glued to or pressed against the interior side of the frame top cover 110 to ensure a tight sealing without leaking between the frame 108 of the device 100, 300 and the sample pad 130. This non-leaking feature is important to ensure consistent fluid flow in the assay chip 104 and consistent fluid front location when fluid flow stops after sample dispense. The front edge of the sample pad 130 (the right side shown in FIG. 1) is preferably perpendicular to the longitudinal direction of the assay chip 104 to provide a boundary condition for better fluid front uniformity as fluid flows to the capture zone 160.

It is preferable to have a relatively fast flow rate (e.g., dispensed sample fluid completely saturate the sample pad in 5 seconds or less) for multiple considerations:
(1) Fluid can be absorbed quickly from the fluid delivery device (e.g., the pipette) which is beneficial for higher throughput design.
(2) Since the sample pad 130 serves as fluid supply for the downstream flow, a fast flow rate in the sample pad 130 ensures a full saturation of the pad 130 (a defined geometry) before fluid flows into the downstream chip structures, promoting a more uniform fluid flow front as sample flows though the conjugate and capture zones 150, 160.
(3) A fully saturated sample pad 130 creates a consistent boundary condition for fluid flow, reducing variations from device to device.

A faster sample pad flow can be achieved for the advantages noted above using a porous material with larger permeability and smaller contact angle (e.g., fiber glass).

Advantageously, the sample pad 130 can also be used to separate the blood cells or large particles from a whole blood sample, while allowing plasma or serum passage.

Moreover and if blood lysis reagent (e.g., with EDTA or heparin) is coated in the sample pad 130, the pad can lysis red blood cells and block cell membrane passage through the pad 130.

### The Conjugate Zone:

In the conjugate zone 150, labeled conjugate is coated by gravure, spray, inkjet, or fluid deposition to ensure a quick and complete dissolution and release of the dried conjugate as sample fluid flows from the sample zone 140 to the conjugate zone 150 downstream. The buffer deposited to the conjugate zone 150 may have more than one kind of labeled conjugates with the same or different labels for multiplexing with multiple capture zones (multiple capture antibodies). The labeled conjugates are assay specific. The labels can be fluorescent dyes, gold nano-particles, microbeads with fluorescent dyes, or magnetic beads. According to one design option, the conjugate zone 150 is located downstream outside the sample zone 140 (as shown in the device 100 of FIG. 1(a) and device 300 of FIG. 3. Fluid arrives at the conjugate zone 150 through the porous structure of the assay chip 104.

After liquid sample is dispensed to the sample zone 140, the sample flows from the sample zone 140 to the conjugate zone 150, dissolves the conjugate 154, and moves downstream to the capture zone 160. With reference to FIG. 8, the dissolved conjugate concentration is typically non-uniform with a peak 153 near the wet front, followed by a long decreasing tail as shown in the exemplary scanned profile obtained of conjugate signal versus location. This figure depicts that given an identical offset, a wider dissolved conjugate distribution 151A (i.e., a smaller spatial gradient of conjugate concentration near the peak 153) along the assay chip 104 has a smaller signal (i.e., conjugate concentration) change, as compared to that from a narrower conjugate distribution 151B (i.e., a larger spatial gradient of conjugate concentration near the peak 153). It is desirable to generate a conjugate concentration distribution with a small spatial gradient for better precision.

According to the current invention, a sample volume is chosen such that fluid flow stops after the peak of the dissolved conjugate reaches the desired location (e.g., inside the capture zone 160 or downstream slightly after the capture zone 160) while the waste zone 170 is still at least partially dry. Fluid flow will resume when the second (e.g., wash) fluid is added to the sample zone 140 (or at another location upstream relative to the capture zone 160) to remove the unbound conjugate from the capture zone 160.

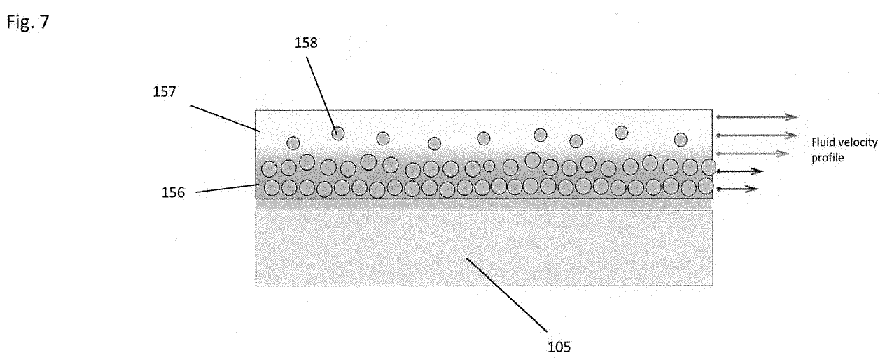

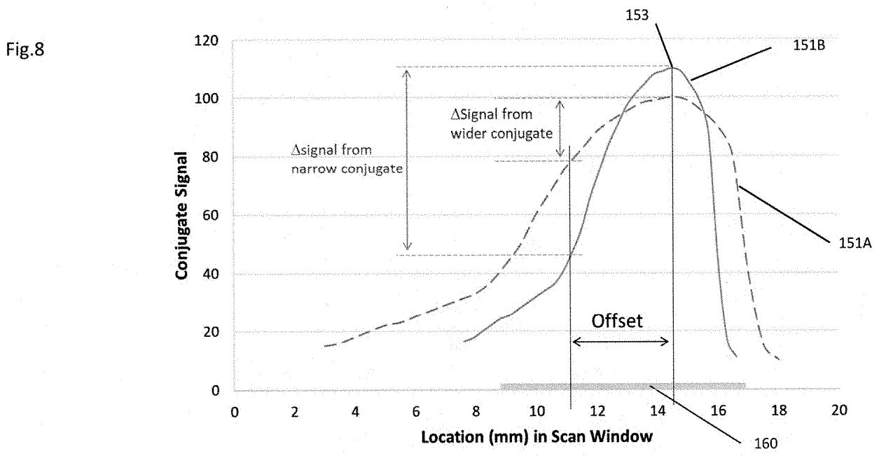

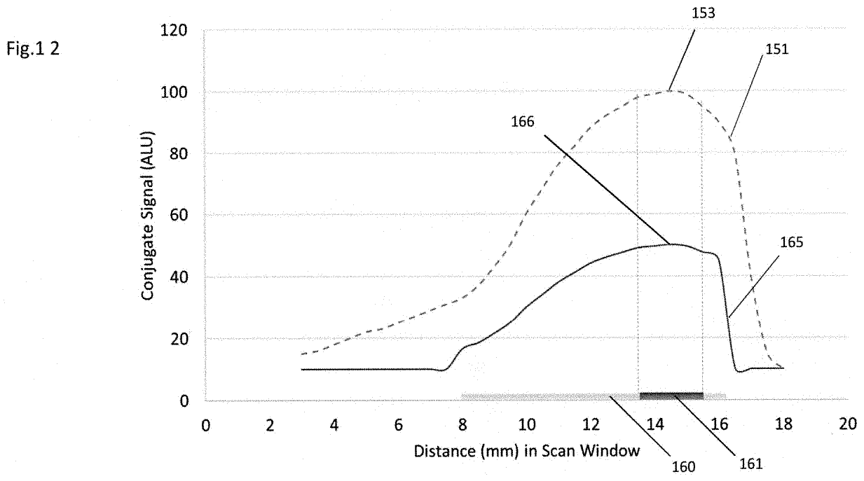

A read area 161 is a segment within the capture zone 160. The size of the read area 161 can be equal to or smaller than the length of the capture zone 160 along the flow direction. When the size of the read area 161 is smaller than the length of the capture zone 160, it is desirable to select a read location in which the spatial gradient is smallest for the dissolved conjugate since the variation of conjugate concentration in the read area 161 at the capture zone 160 affects assay precision. FIG. 12 illustrates that the read location is centered at the peak conjugate concentration location 153 prior to wash, where the spatial gradient is the smallest. Curve 151 shows the dissolved conjugate distribution prior to wash. Curve 165 shows the bound conjugate after wash is complete. For a larger (i.e., longer) capture zone (e.g., 8 mm), the read area 161 is preferably a segment (e.g., 1.5 mm) of the capture zone 160 centered at the peak location of the total conjugate prior to wash. In theory, any location within the capture zone 160 can be the read location, as long as the read area is inside the capture zone 160 and the total conjugate signal prior to wash is sufficiently high as compared to the baseline signal (i.e., the sample wetted chip background signal without conjugate). In practice, it is preferable to use the peak location 153 of the total conjugate signal (both free and bound prior to wash) as the read location for better consistency in conjugate concentration among reps since peak location varies with sample fluid volume. It is important to detect the edges of the capture zone 160 to ensure the read area is inside the capture zone 160. If the peak location is outside the capture zone 160 by a pre-defined distance threshold, the test will not be used to predict analyte concentration. To reduce the sensitivity to read location, it is desirable to reduce the spatial gradient of dissolved conjugate along the capture zone 160 and make the conjugate spread wider as the solution flows along the chip 104 to the capture zone 160. This can be achieved by making the porous medium of the assay chip 104 into two separate layers 156, 157 with smaller pore size in one of the layers 156, while keeping the larger pore size in the other layer 157 across the thickness of the porous structure in the conjugate zone 150. This “two-layered” porous structure shown in FIG. 7 has larger velocity differences across the thickness of the pore structure, spreading the dissolved conjugate over a wider range along the assay chip 104.

One way to make a smaller pore size layer is to trap beads inside the porous structure by depositing suspensions of beads 158 (e.g., ˜2-micron in size, depending on the pore size of the porous layer 106 in the chip 104) from the top side of the porous structure in the conjugate zone 150. To create a chip 104 with beads 158 and conjugates 154 in the conjugate zone 150 and antibody coated beads 162 in the capture zone 160, the major steps are: 1) Deposit the beads 158 to the conjugate zone 150 in the top side of the porous layer 106 of the chip 104 and dry; 2) Deposit antibody coated beads 162 to the capture zone 160 in the top side of the porous layer 106 and dry; 3) Deposit labeled conjugate 154 to the conjugate zone 150 in the top side of the porous layer 106 and dry; 4) Attach the support layer 105 to the top side of the porous layer 106. Here the support layer 105 is optional. After manufacture, the chip 104 is flipped upside-down with the support layer 105 beneath the porous layer 106. As shown in FIG. 7, the suspension of the beads 158 will stay mostly near the surface of deposition (i.e., near the bottom following the flipping of the chip 104) with higher bead density due to trapping effects. The beads 158 in the conjugate zone 150 are hydrophilic without non-specific binding capabilities. This makes fluid flow slower than the other side without beads (or with lower bead density), and resulting a slower release of the labeled conjugate, and resulting in a wider conjugate distribution along the capture zone 160. Accordingly, the pore size becomes smaller with the trapped beads 158 in this layer 156, leading to slower fluid flow velocity as compared to the other layer 157 across the thickness of the conjugate zone 150 without beads or having a lower bead density. The velocity differences between the layers 156, 157 across the thickness leads to a wider conjugate distribution and a smaller concentration gradient along the capture zone 160.

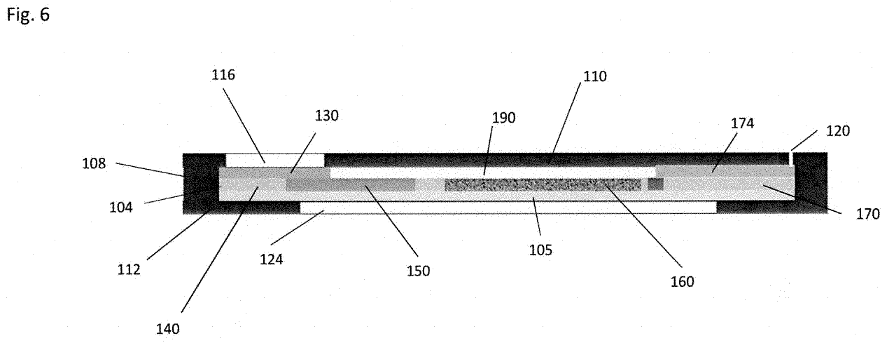

In another design, the sample pad 130 can be partially or completely disposed above the conjugate zone 150 in the assay chip 104. FIG. 6 depicts a version in which the conjugate zone 150 is partially beneath the sample pad 130 for a wider conjugate concentration profile as conjugate flows to the capture zone 160. This device design leads to a slower and delayed release of conjugate, since fluid flow velocity is highest in the front edge and slower to the opposite side of the sample pad 130 over the conjugate.

### The Capture Zone:

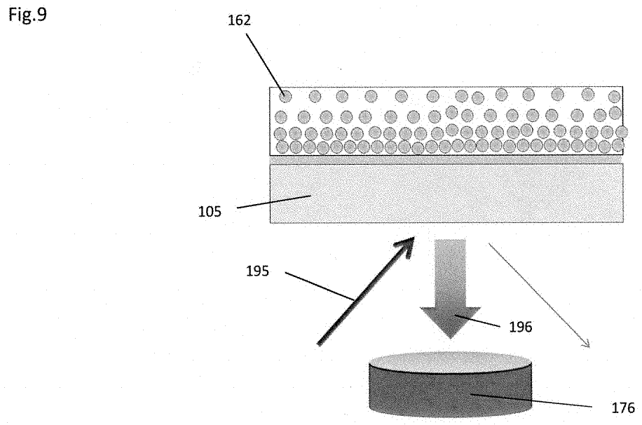

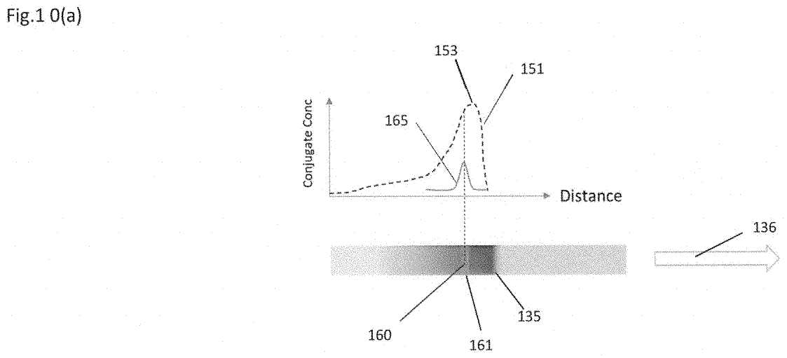

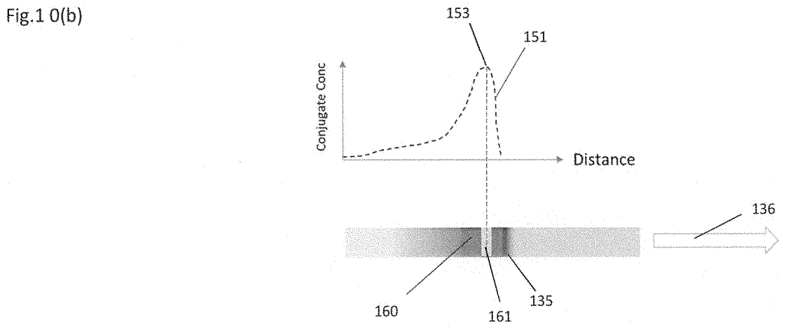

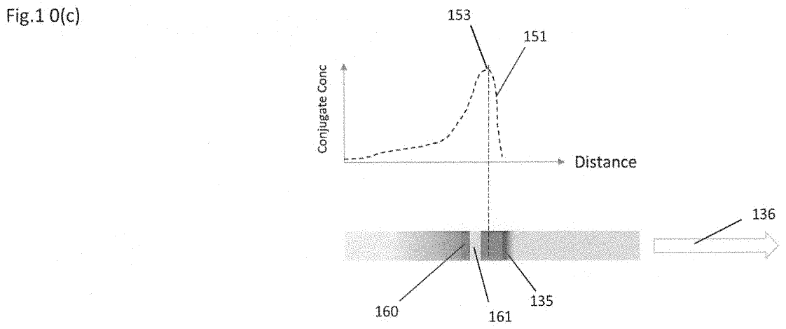

The capture zone(s) 160 in the device according to at least one version of the current invention are built by trapping smaller beads (e.g., ˜1.8-microns polystyrene beads) with surface coated antibody into the pores of the chip with larger pore sizes (e.g., fiber glass filters, or films created by piles of ˜10 micron-beads, etc.). FIG. 9 illustrates an enlarged schematic side view of a portion of an exemplary lateral flow device in accordance with aspects of the invention and more specifically an exemplary capture zone having antibody coated beads 162 trapped inside fiber glass (or other porous or capillary structures). The optional chip support layer 105 (a transparent solid structure) is glued to the porous structure in this particular design. If the whole chip structure is a molded solid (e.g., plastics) with capillaries, the chip is just a single integrated section without the need for glue. A bead density gradient due to bead trapping in manufacture process is expected. The side having a higher bead density should face the optical sensor 176 for better assay sensitivity. The excitation light 195 with wavelength λ₁ is at an angle (e.g., 45 degrees) to the chip surface. The optical sensor 176 is disposed orthogonal to the chip surface in order to receive the emission light at wavelength λ₂.

In the manufacture process, the beads are coated with assay specific antibody and the surface is blocked by protein (e.g., BSA) and/or surfactant to avoid non-specific binding. The coated beads 162 are then washed to remove any unbound material. This method creates a mono layer of antibody on the bead surface with minimum chance for non-specific binding. To create the capture zone 160, the antibody coated beads 162 in a buffer suspension are deposited to the porous structure of the assay chip 104 and then dried. One advantage of this method is that there is little “coffee ring” effect for the coated beads 162 due to the trapping of the porous structure. Another advantage is that the coated beads 162 inside the assay chip 104 are trapped without movement in an assay process with fluid flow. A third advantage is the increased surface area of the coated antibody and reduced diffusion distance for analyte and conjugate, enhancing sensitivity and improving capture efficiency for analyte and conjugate compared to the larger porous structure without the beads 162.

The width of the capture zone 160 is determined by the deposition parameters, such as coating fluid coverage area and flow rate, solid ratio of the coated beads 162 in fluid suspension, and the porous medium's linear feeding speed in the coating process.

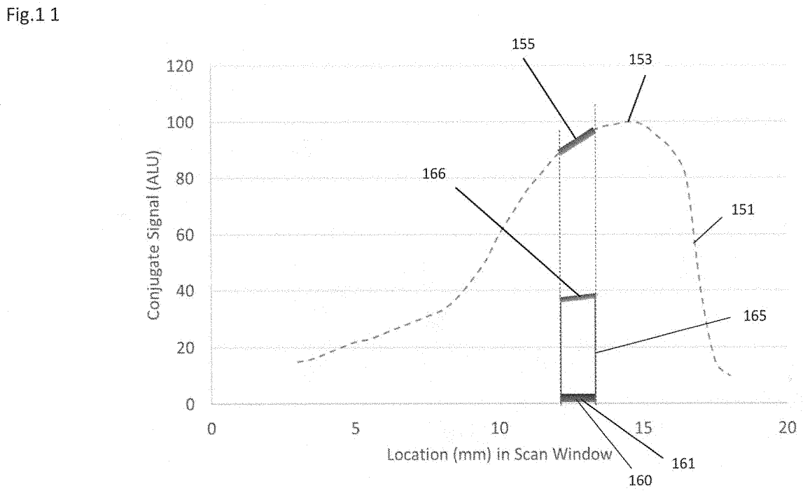

There are at least two (2) types of capture zone designs (short and long) with different ways to “find” the assay signal read location. FIGS. 10(a)-10(c) depict scanned conjugate profiles of exemplary lateral flow devices having a read location and read area for short or long capture zones, respectively, of an exemplary device. The scanned conjugate profile shown in FIG. 10(a) has a short capture zone. The conjugate concentration profile 151 is obtained before wash fluid is applied to the device, the profile 151 having a peak 153 located at the right of the capture zone 160. The scanned conjugate concentration profile 165 is taken after the application of wash fluid to the device. The wet-dry interface 135 is at the right of the capture zone 160 during assay incubation. Fluid flows from left to right as shown by arrow 136. The read location and read area 161 are exactly the same as the capture zone 160.

FIGS. 10(b) and 10(c) show conjugate profiles having a long capture zone 160. FIG. 10(b) shows a smaller read area 161 than the longer capture zone 160 at the peak location of the conjugate profile 151 prior to wash. Fluid flows from left to right as shown by arrow 136. The wet-dry interface 135 is at the right of the capture zone 160 during assay incubation. FIG. 10(c) shows a smaller read area 161 at a fixed distance from the peak location 153 of the conjugate profile 151 prior to wash. Fluid flows from left to right as shown by arrow 136. The wet-dry interface 135 is at the right of the capture zone 160 during assay incubation. For a short capture zone (e.g., <1.5 mm), the read location is exactly the capture zone location and the read area is typically the whole capture zone (FIG. 10(a)). For a long capture zone (e.g., >1.5 mm, preferably >3 mm), the size of read area is typically shorter than the length of the capture zone 160 and the read location is preferably at the peak location (FIG. 10(b)) within the capture zone 160 for a smallest spatial gradient in the total concentration profile prior to wash, or at a fixed offset from the peak location (FIG. 10(c)) of the total concentration prior to wash.

For the short capture zone (e.g., <1.5 mm) design with the entire capture zone 160 as the read area 161, the read location of the read area 161 should be determined based on the design geometry (a known parameter) or based on bound conjugate signal 165 after wash is complete. To detect the read location (the capture zone) with bound conjugate signal 165 after wash, either the peak of the scanned signal 166 or the two edges of the scanned signal 165 can be used as shown in FIG. 11. FIG. 11 illustrates a total conjugate concentration profile 151 of an exemplary lateral flow device in accordance with aspects of the invention, as taken prior to wash and bound conjugate 165 after wash for a narrow capture zone 160. The length of a narrow capture zone 160 is typically shorter than 1.5 mm. The scanned signal of the bound conjugate 165 is used to determine the read location (also the capture zone location). The read area 161 for a narrow capture zone 160 is typically the whole capture zone. The signal 155 of the total conjugate in the read area 161 prior to wash is used for scaling. The scanned signal 166 after wash in the read area 161 is the bound signal to be scaled. The distance between the peak location 153 of the signal prior to wash and the detected capture zone edges of 165 after wash are used for quality control. If the distance is beyond a given threshold, the test may be aborted. After determining the read location 161, the signal in the read area with total conjugate (both free and bound) prior to wash is used for signal scaling and the signal 166 in read area after wash is scaled.

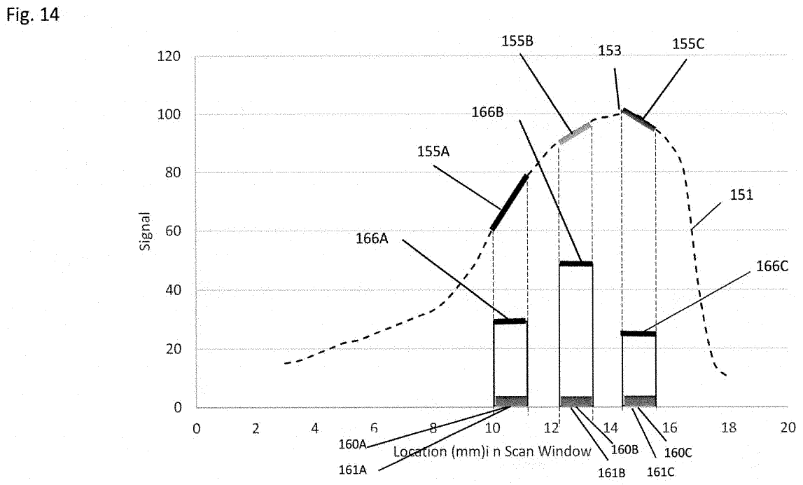

The advantage of a short capture zone design is space availability for multiplexing (multiple assays in one chip) in a very small device, as illustrated in FIG. 14 having three (3) capture zones 160A, 160B, 160C. The three read areas 161A, 161B, 161C correspond to the three capture zones 160A, 160B, 160C separately and the read location of each capture zone 160A, 160B, 160C should be detected separately similar to the one capture zone device design (FIG. 11) described previously. FIG. 14 illustrates conjugate signal profiles 151 for an exemplary lateral flow device having multi-capture zones 160A, 160B, 160C in which the conjugate peak location prior to wash and after sample flow stops is detected for quality assurance. The bound conjugate signal 155A, 155B, 155C is obtained after wash is complete. The conjugate signal 155A, 155B, 155C corresponding to each capture zone 160A, 160B, 160C prior to wash will be used for scaling that capture zone. It should be noted that the number of capture zones can be easily varied, meaning more or less than the three zones that are illustrated.

The disadvantage of this design is that the capture zone may experience large conjugate concentration variations during incubation caused by variations in sample fluid volume, flow rate, and/or device manufacture.

For a larger (i.e., longer) capture zone (e.g., 8 mm), the read area can be a segment of capture zone 160, as shown in FIG. 12. While any location within the capture zone 160 can be the read location as long as the read area 161 is always inside the capture zone 160, it is preferable to use the peak location 153 as the read location to accommodate peak location variations with smaller concentration gradients. The peak location can be detected by the total conjugate (both free and bound) prior to wash, or by the bound conjugate peak 166 after wash.

The read location can be exactly the peak location (the smallest gradient in conjugate concentration as shown in FIG. 10(b)) or alternatively defined as an off-set from the peak location, as discussed previously according to FIG. 10(c). The off-set is assay specific and is optimized through experiments.

The advantage for a wide capture zone is that there are no edge effects from coated antibody beads since the read area is within the larger capture zone. Another advantage is that it is less sensitive to variation in fluid volume, flow, and device manufacture.

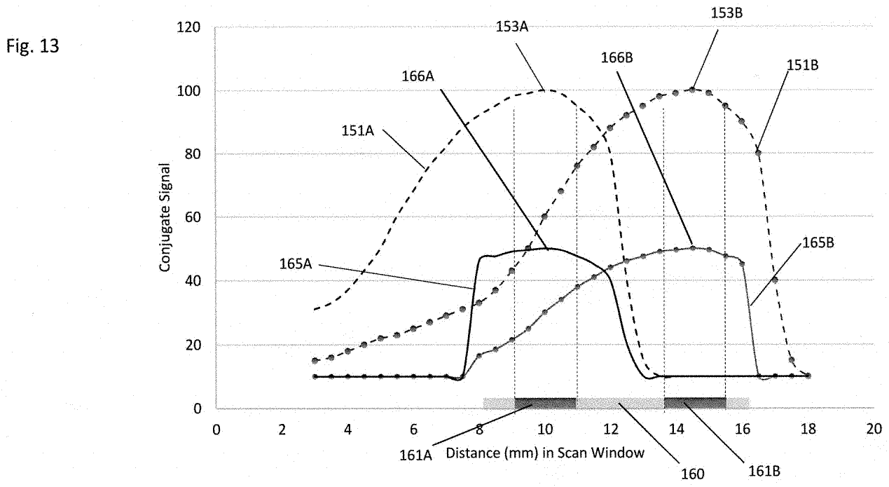

FIG. 13 depicts a pair of conjugate profiles 151A, 151B illustrating shifts occurring due to differences in sample volumes. As shown, the peak conjugate location 153B flows further downstream in the chip for a sample with a larger volume. Within a wider capture zone 160 (e.g., 8 mm), the conjugate concentrations may vary significantly among the reps at any given location, but the peak conjugate concentrations 153A, 153B have small variation among the reps within the capture zone. Therefore, it is advantageous to use the signal peak location of total conjugate as the read location. As shown in FIG. 13, although the conjugate peak location 153A, 153B differs by about ˜4.3 mm between the two devices (due to imprecision of dispensed sample volume or device manufacture), the two devices experience about the same conjugate concentration for incubation with the detected peak as read location. This reduces assay's sensitivity to sample volume or manufacture variabilities.

A disadvantage for the large capture zone design is that less space is available for multiplexing inside the small chip.

In summary, read locations can be determined with the following methods:
a) Based on the design geometry of the device with known read location.
b) For a short capture zone (i.e., less than 1.5 mm), the read location is the corresponding peak in the scanned profile after wash is complete with bound conjugate.
c) For a short capture zone (i.e., less than 1.5 mm), the read location is defined between the two edges of the scanned signal above the background after wash is complete with bound conjugate.
d) For a long capture zone (i.e., more than 1.5 mm), the peak location of total conjugate prior to wash is detected first. The peak location is used as a reference location for the read location. The read location is either exactly the peak location, or a location with a predefined distance to the peak location. The edges of the capture zone are determined using the scanned signal above background after wash is complete. The read location shall be within the capture zone.
e) For a long capture zone (i.e., more than 1.5 mm), the peak location of the bound conjugate after wash is detected. This peak location is used as a reference location for the read location. The read location is either exactly the peak location, or a location with a predefined distance to the peak location. The edges of the capture zone are determined using the scanned signal above background after wash is complete. The read location shall be within the capture zone.

### The Waste Zone:

The waste zone 170 includes a hydrophilic porous pad 174 sitting above the assay chip, or alternatively is part of the chip structure (either fiber glass, solid capillary structure, or beaded structure) downstream to the capture zone 160. The waste zone 170 is used to absorb sufficient amount of fluid such that the unbound conjugate in the read areas of the capture zone 160 is sufficiently washed off from the read area with the addition of the second (e.g., wash) fluid before the waste zone 170 is fully saturated and fluid flow stops. To allow for second fluid addition, the waste zone 170 is at least partially dry during assay incubation. The capillary force in the absorption pad 174 is preferably smaller than or equal to that in the sample pad 130 so that fluid flow will stop when sample is fully embedded into the pore structure of the sample zone 140.

### Scaling Signal to Improve Assay Precision:

After the lateral flow device 100, 300 is inserted into the incubator of a high throughput testing instrument for incubation at specified temperature, the device moves relative to an optical system of a contained scanning instrument, such as a fluorimeter, along the direction of the scan window 124. Two different designs are herein described to achieve the relative movement between the sensor and the device scanning window 124. It will be understood that there are also variants of these designs to one of skill in the field. According to one design, the optical sensor is stationary, while the lateral flow device moves with the incubator (not shown) such that the scan window 124 passes though the stationary optical system. According to a second design, the device inside the incubator is stationary or fixed, while the optical sensor moves along the scan window 124.

The assay response could be defined as the scanned signal (the peak value, the medium, the mean, or the sum of signal inside the area) in the read area. The assay response is used to create a dose response curve with assay calibrators. The calibration curve can then be used to predict analyte concentration with assay response from the sample analyte. One problem is that in the read area at the detected or pre-defined read location, the conjugate concentration is affected by sample volume, flow and manufacture variations and the variation leads to bound signal variation. That bound signal variation leads to assay imprecision in predicted analyte concentration.

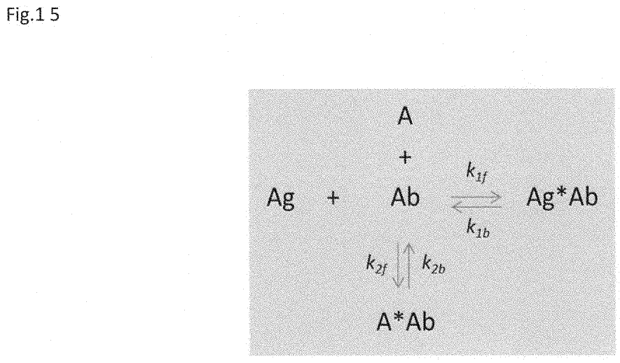

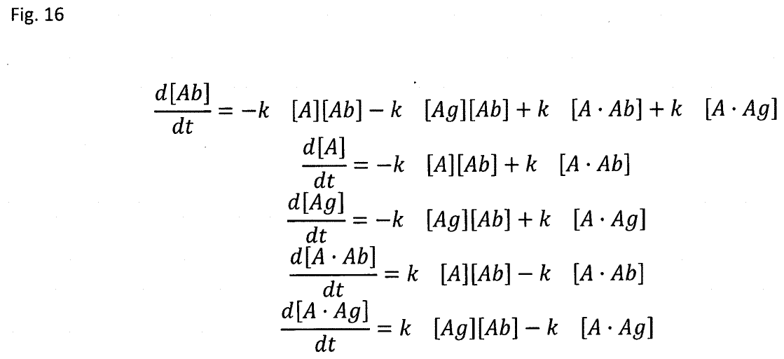

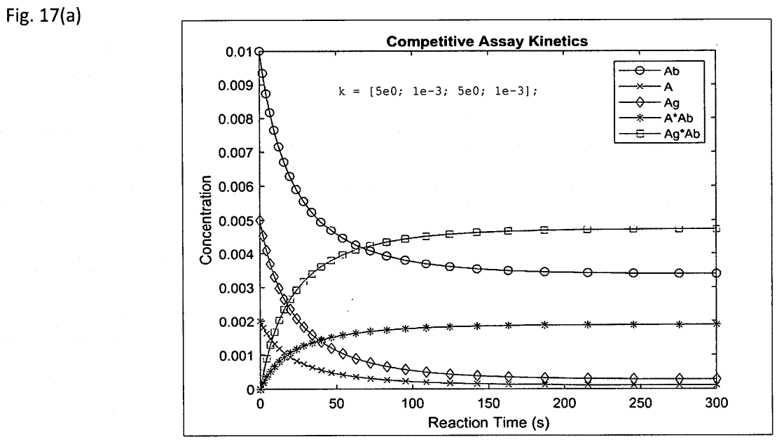

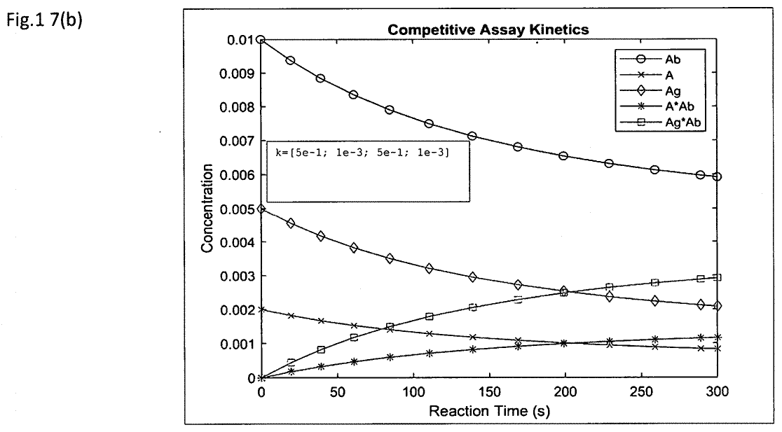

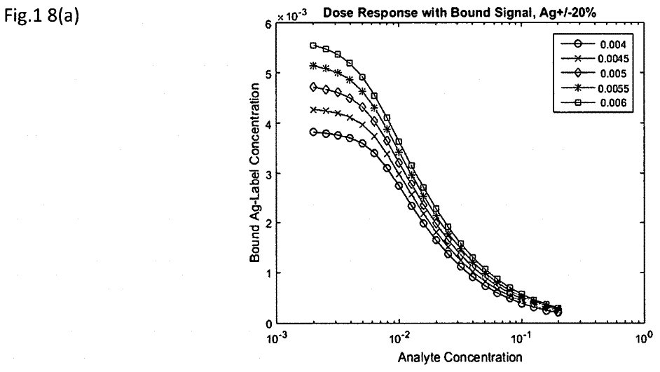

The effects of conjugate concentration on analyte can be studied with a kinetics model, which is shown in FIG. 15. The model shown is for a competitive assay with labeled conjugate (Ag), the capture antibody (Ab), and the analyte in the sample (A) with immunocomplex of Ag-Ab and A-Ab. k_{1f} and k_{1b} are forward and backward kinetics rate constants for Ag and Ab. k_{2f} and k_{2b} are forward and backward kinetics rate constants for A and Ab. FIG. 16 is the coupled ordinary differential equations for the competitive assay kinetics. FIGS. 17(a) and 17(b) show the predicted concentrations for all reactants as a function of time for two different cases. One is a faster kinetics (k_{1f}=5 M⁻¹S⁻¹; k_{1b}=0.001 S⁻¹; k_{2f}=k_{2b}=0.001 S⁻¹) and the kinetics reaction reaches equilibrium within 300 seconds. The other one is a slower kinetics (k₁f=0.5 M⁻¹S⁻¹; k_{1b}=0.001 S⁻¹; k_{2f}=0.5 M⁻¹S⁻¹; k_{2b}=0.001 S⁻¹) and the reaction is not in equilibrium yet at 300 seconds.

The initial concentrations for both cases are the same—the bound analyte (A*Ab) and bound conjugate (Ag*Ab) concentrations are zero initially. The initial analyte (A) concentration is 0.002M. Initial labeled conjugate (Ag) concentration is 0.005M, and the initial capture antibody (Ab) concentration is 0.01M.

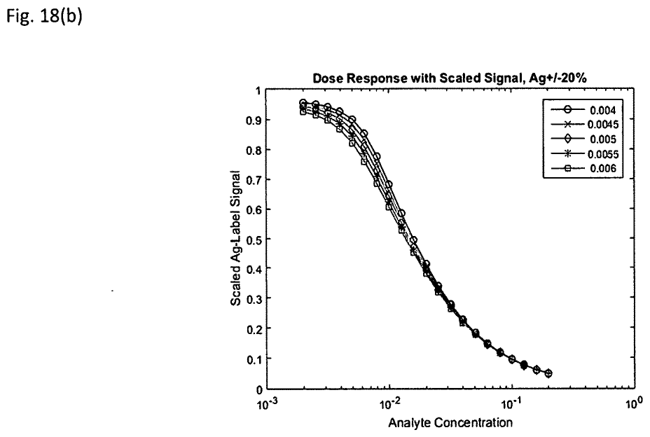

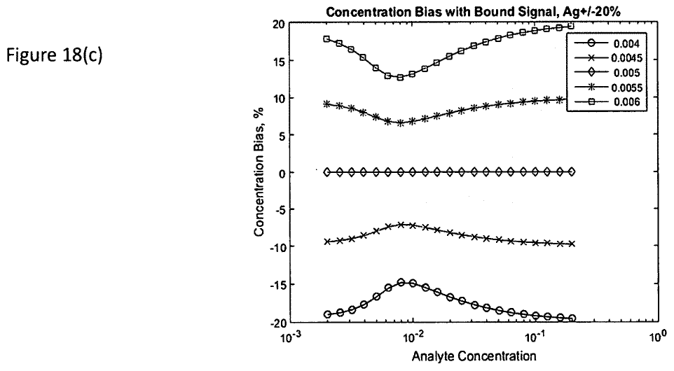

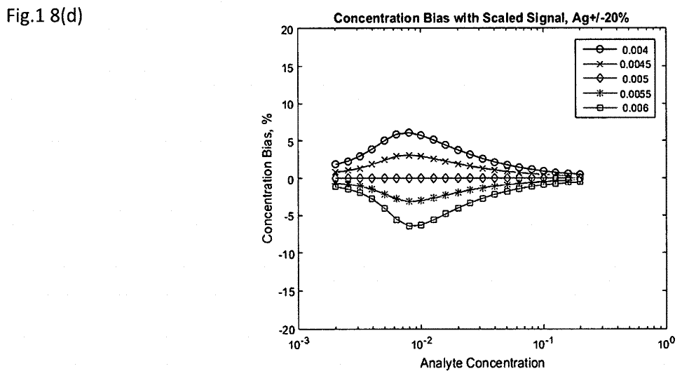

FIGS. 18(a)-18(d) depict graphical profiles indicative of dose response for faster competitive assays and predicated concentration bias for faster competitive assays in accordance with aspects of the invention represents scaling effects on assay precision for faster kinetics of a competitive assay. When the assay response is defined as the bound signal scaled by the total conjugate in the read area, the predicted concentration bias is much smaller. FIG. 18(a) shows the dose response curves when initial conjugate concentration deviates from the 0.005M by as much as 20%, i.e., to 0.004M in the low end and 0.006M at the high end. For the same analyte (A), the bound conjugate (label-Ag) is affected by the initial free conjugate (Ag) concentration. For example, if initial free conjugate (Ag) concentration is changed to 0.006M from 0.005M, the bound conjugate concentration is higher. As a result, the predicted concentration with the standard calibration curve (obtained with free conjugate at 0.005M) will be lower. FIG. 18(c) shows the percentage differences in predicted analyte concentration due to initial conjugate concentration deviation from the standard 0.005M. For +/−20% differences in initial free conjugate concentration from 0.005M, the predicted concentration will differ by up to +/−20% as well.

The performance with the scaled signal as assay response is shown in FIGS. 18(b) and 18(d) for the faster kinetics. The scaled signal is the signal after wash scaled by the signal prior to wash (the normalization value described previously) in the read area. The same +/−20% differences in initial free conjugate concentration from 0.005M leads to a much smaller differences in the dose response curve shown in FIG. 18(b). The predicted concentration difference is less than 6% as shown in the dose response curve of FIG. 18(d), much better than the 20% obtained with the scanned signal itself.

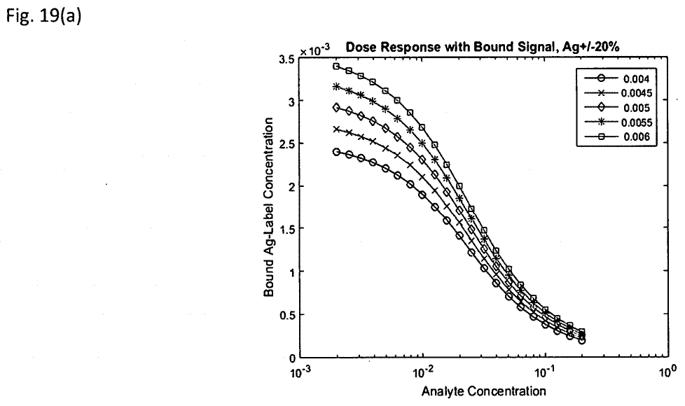

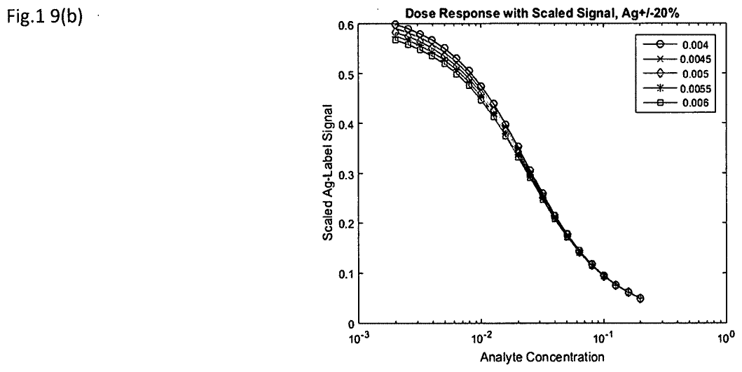

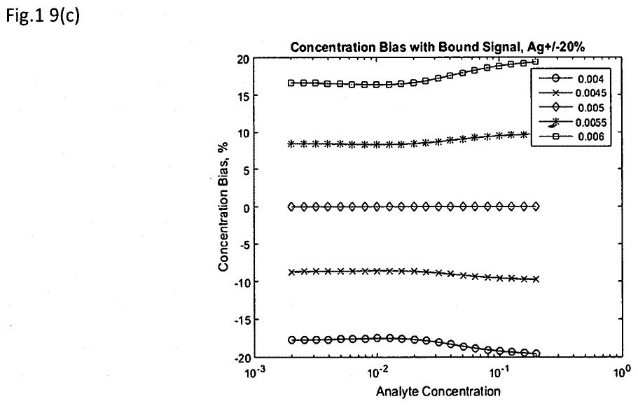

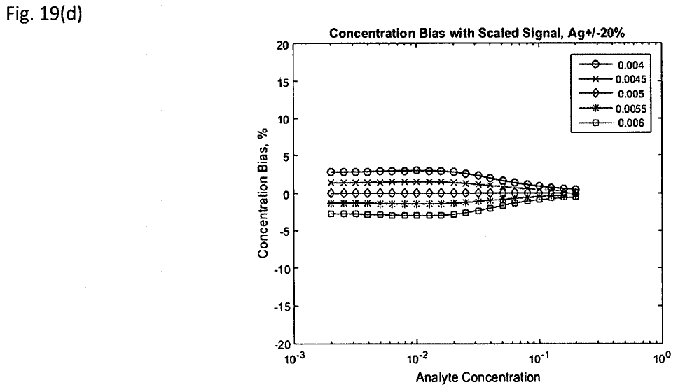

For a slower kinetics as shown in FIGS. 19(a)-19(d), the predicted analyte concentration for scaled signal differs from the true concentration by less than 4%, a dramatic improvement over the original signal with up to 20% differences.

The method according to this invention performs scaling as described and defines the scaled signal as the assay response. Again, the assay response is the bound conjugate signal after wash scaled by the total conjugate signal prior to wash in the read area. The signals can be the peak, the mean, the medium, or the sum of the scanned signal in the read area. The read area is predefined with a known dimension (e.g., 1 mm width by 1 mm) at the read location.

Error Detection with Conjugate Profiles:

The scanned profiles are interrogated to determine if the device is performing normally. An erratic profile may indicate errors in fluid flow, manufacture, or other issues. An alert should be issued and the result may be aborted for analyte concentration prediction. For example,
a) If the absolute value of the spatial gradient in the read location is larger than a specific threshold, report an error (conjugate error, many potential causes).
b) If the distance between predicted read location and the default read location is larger than a specific threshold, report an error (sample volume error).
c) If the conjugate signal at read location prior to wash is lower than a specified threshold, report an error (conjugate or sample volume error).
d) If peak location is detected beyond a predefined location threshold after wash, report an error.

### Device Design Options:

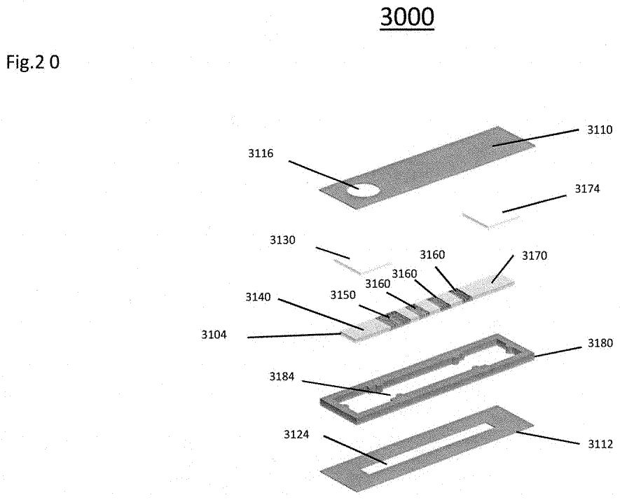

Various other device designs can be contemplated that employ features in accordance with the invention. For example, FIG. 20 shows a lateral flow device 3000 defined by a single assay chip having three (3) separate capture zones 3160 (i.e., three different capture antibodies). The adjacent conjugate zone 3150 according to this embodiment is deposited with a mixture with three different conjugates corresponding to the three capture antibodies separately for three different assays. The size of each capture zone 3160 is shorter compared to the design 300 shown in FIG. 3, wherein each of the other components are basically the same between the two devices 300, 3000 shown in FIG. 3 and FIG. 20, respectively. More specifically, the lateral flow device 3000 is similarly defined by a solid frame 3108 that includes a top cover 3110 and a bottom cover 3112, with an assay chip 3104 disposed within the interior of the frame 3108. A sample pad 3130 is fluidically connected to the assay chip 3104 at one end of the chip, defining a sample area or zone 3140 aligned with a sample metering window 3116 formed in the top cover 3110. An absorption pad 3174 is fluidically connected to the assay chip 3104 at an opposing end of the device 3000 defining a waste zone 3170. The device 3000 according to this embodiment further includes a spacer structure 3180 having nubs 3184 or equivalent structure that maintains the assay chip 3104 specifically and in fixed relation. An air vent (not shown) can also be included as an optional feature.

For multiple capture zones 3160 as shown in the device of FIG. 20, the peak location of total conjugate prior to wash (second fluid) addition is used for quality assurance. If the peak location is beyond specified limits (e.g., not detectable, or exceed a specified distance from the capture zone 3160), the assay results may be abandoned. The read area for each capture zone 3160 of this multi capture zone design is typically the corresponding capture zone area. The location of each capture zone 3160 is determined with conjugate signals after wash with either signal peak or the two edges of the signal. FIG. 14 illustrates captured conjugate in each of the three capture zones 3160 after wash, as well as the total conjugate prior to wash.

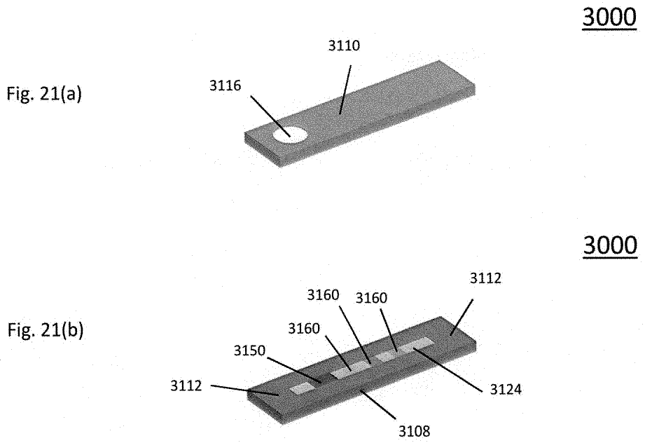

FIGS. 21(a) and 21(b) show respective top and bottom perspective views of the lateral flow device 3000. According to this embodiment, the fluid metering window 3116 formed in the top cover 3110 of the device 3000 is provided for fluid metering to the sample pad 3130 in the sample zone 3140. A scan window 3124, which according to this embodiment, is formed in the bottom cover 3112 of the device frame 3108 exposes at least a portion of the conjugate zone 3150 and all the capture zones 3160. It will be understood that the number of capture zones 3160 can be suitably varied.

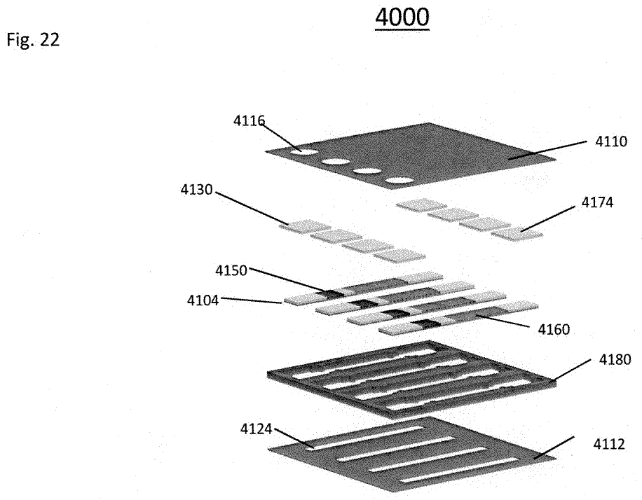

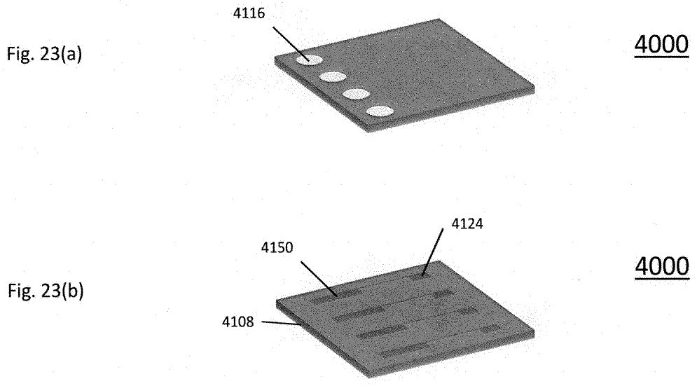

Still other variations are possible within the herein described inventive aspects. For example, FIGS. 22, 23(a) and 23(b) show an exemplary lateral flow device 4000 having four (4) identical chips 4104 disposed within a common frame 4108. As in the preceding design, the frame 4108 is made up of a top cover 4110 and a bottom cover 4112, defining an interior. In addition to the assay chips 4104, corresponding sample and absorption pads 4130, 4174 are further provided, thereby defining separate and independent chip and pad sets. Each of the assay chips 4104 and the associated pads 4130, 4174 are isolated from other chip and pad sets of the herein described device 4000. That is, there is no liquid communication between the chip and pad sets of this device 4000. The herein described lateral flow device 4000 is similarly constructed to include a spacer structure 4180 in addition to the sample pads 4130, the absorption pads 4174, and assay chips 4104, each of which are similar in design and features to those depicted and described in the lateral flow device 300 of FIG. 3. According to this device embodiment, each chip 4104 is separated from the other chips 4104 by the hydrophobic spacer structure 4180. Each assay chip 4104 according to this version has its own fluid metering window 4116 and scanning window 4124 provided in the top cover 4110 and bottom cover 4112, respectively. An optional air vent (not shown) can also be optionally provided. The sample pad 4130 and absorption pad 4174 for each chip 4104 are also separated from pads of the other chip and pad sets with void spaces being provided therebetween. In effect, the lateral flow device 4000 according to this embodiment is analogous to having four identical devices 300, such as shown in FIG. 3, assembled into a single device. The herein described lateral flow device 4000 is configured to run four different samples with one identical test each. The device can also run four reps for the same sample. Either a scanned image or four individual scans can be performed to obtain the assay signal through the four scan windows 4124.

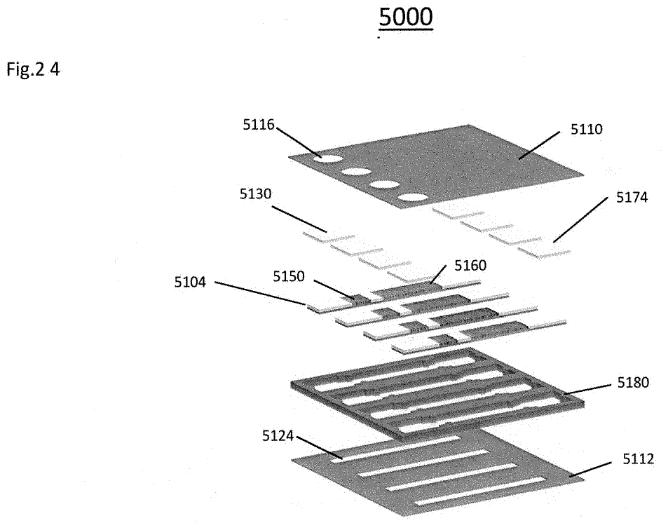

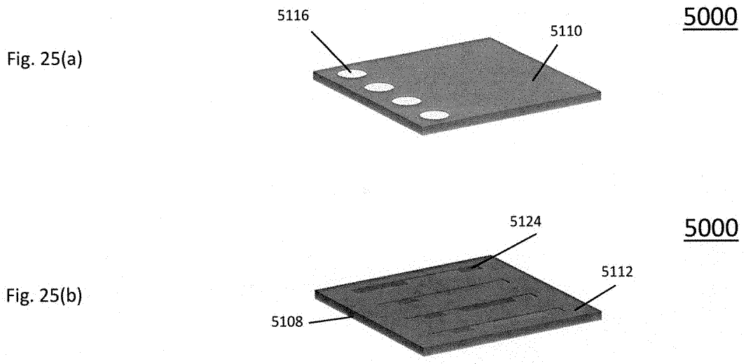

Another exemplary device 5000 shown in FIG. 24 and FIGS. 25(a) and 25(b) has four chips 5104 as well as sample and absorption pads 5130, 5174 defining chip and pad sets, as in the preceding embodiment. All the chips 5104 and the associated pads 5130, 5174 are isolated from other chip and pad sets. More specifically, there is no fluidic communication between them. This device embodiment is also defined by a frame 5108 made up of a top cover 5110 and a bottom cover 5112 in addition to the sample pads 5130, absorption pads 5174, a spacer frame 5180. Each chip 5104 is separated from other chips of the device 5000 by the hydrophobic spacer structure 5180, which is configured to maintain the chips 5104 in spaced and fixed relation. Moreover, each chip 5104 has its own fluid metering window 5116 and scan window 5124 formed according to this embodiment in the top cover 5110 and bottom cover 5112, respectively. The sample pad 5130 and absorption pad 5174 for each chip 5104 are also separated from other pads with void spaces between them. According to this design, there is a total of four (4) different capture antibodies in the device 5000 with one unique antibody in each chip's capture zone 5160 for a unique assay test. Each chip 5104 has its own conjugate zone 5150 corresponding to the assay for the chip. This specifically designed device 5000 can run four different tests with one sample. In operation, four (4) separate sample dispenses are required for this design with each dispense being made at a corresponding sample metering window 5116 of the device 5000. The herein described lateral flow device 5000 is also configured to perform four different tests for four different samples, with one assay test for each sample. Either a scanned image or four individual scans can be performed to obtain the assay signals through the four scan windows 5124.

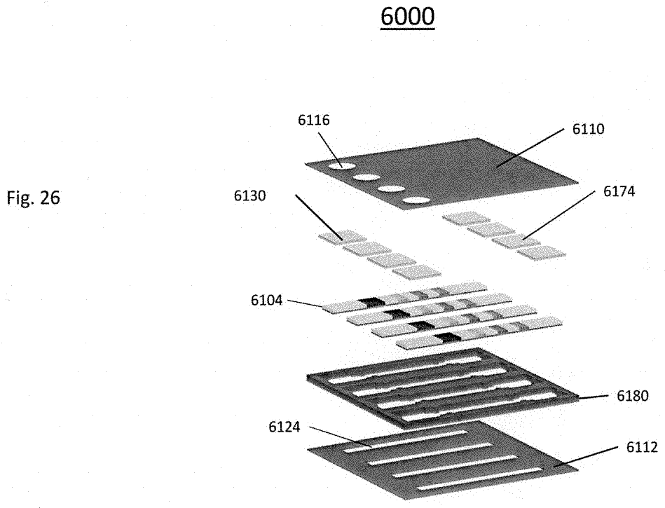

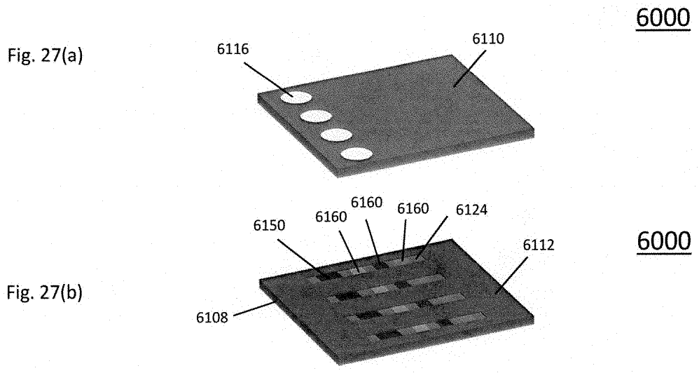

FIG. 26 and FIGS. 27(a) and 27(b) depict yet another exemplary lateral flow device 6000 having four identical chips 6104 disposed in the interior of a solid frame 6108 defined by a top cover 6110 and a bottom cover 6112. Each chip 6104 according to this embodiment can perform three (3) different tests in which one sample pad 6130 and one absorption pad 6174, similar to those previously described in device 3000, are provided for each chip 6104. The top cover 6110 includes four (4) fluid metering windows 6116 formed therein in spaced parallel relation, with each window 6116 corresponding to and aligned with one of the sample pads 6130. The bottom cover 6112 according to this embodiment includes four (4) scan windows 6124, with each scan window 6124 corresponding to one of the chips 6104. All the chips 6104 and the associated pads 6130, 6174 are isolated from the remaining chip and pad sets in spaced relation as supported by a spacer structure 6180, similar to versions previously described. As in the preceding embodiments, there is no liquid communication between the chip and pad sets. This device 6000 as described is configured to run four different samples with three tests for each sample.

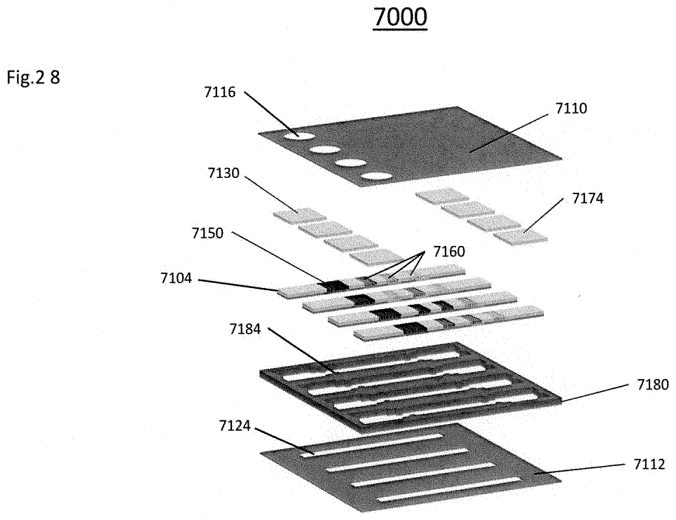

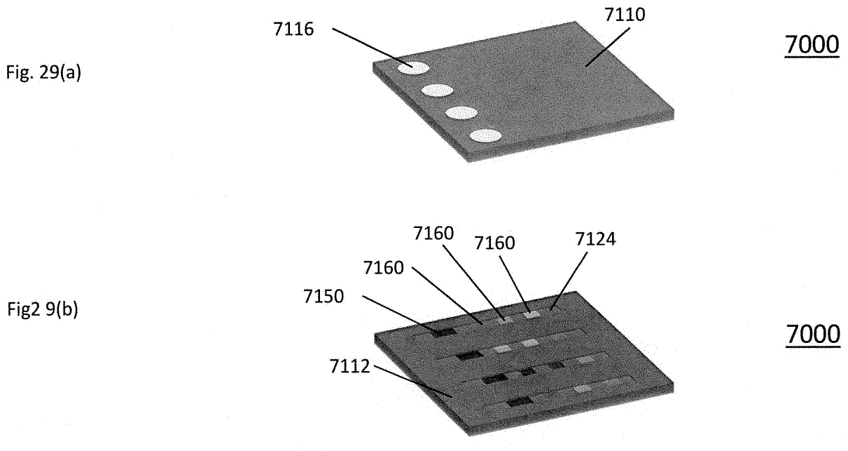

FIG. 28 and FIGS. 29(a) and 29(b) show yet another exemplary lateral flow device variant 7000 having four chips 7130 in a frame 7108 with 3 tests in each chip with a total of 12 different capture zones 7160. This specific device 7000 can perform 12 different tests for one sample. For each of the four chips 7104, there is one sample pad 7130 and one absorption pad 7174 fluidically connected at opposite ends of each chip 7104. The frame 7108 is similarly provided with a top cover 7110 and a bottom cover 7112. According to this version, the top cover 7110 has four (4) fluid metering windows 7116 formed therein and corresponding to each sample pad 7130. Likewise, the bottom cover 7112 has four (4) scan windows 7124 corresponding to each chip 7104 and having a length to permit scanning of at least a portion of defined conjugate and capture zones 7150, 7160 of the device 7000, at a minimum. As in the preceding embodiments, all the chips 7104 and the associated pads 7130, 7174 are isolated from other chip and pad sets with a spacer structure 7180, meaning there is no liquid communication between them.

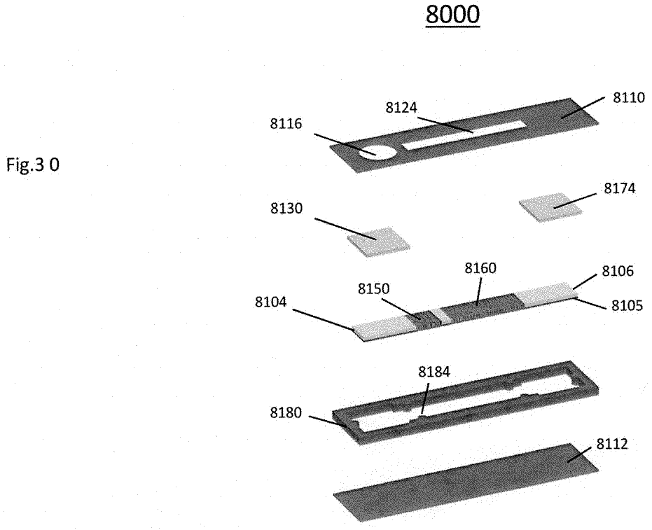

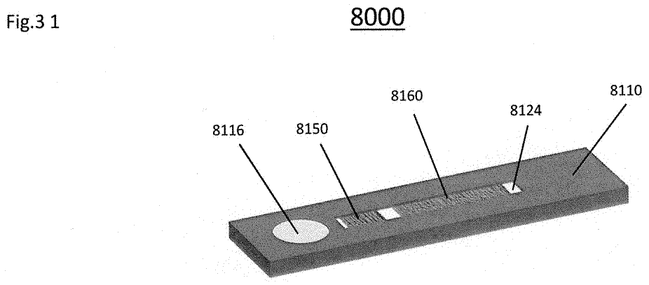

Still other variants to the device design are contemplated. For example, FIGS. 30 and 31 illustrate another exemplary lateral flow device 8000. The device 8000 according to this version is configured with both a fluid metering window 8116 and scan window 8124 being formed in a top cover 8110 of the device 8000. Other than the location of the scanning window 8124, this device 8000 is structural identical to the device 300 shown in FIG. 3.

Similarly, all the herein described lateral flow devices 3000, 4000, 5000, 6000, 7000 and other variants can be similarly configured to have the scanning window(s) alternatively disposed or formed within the top cover of the device in lieu of forming same in the bottom cover.

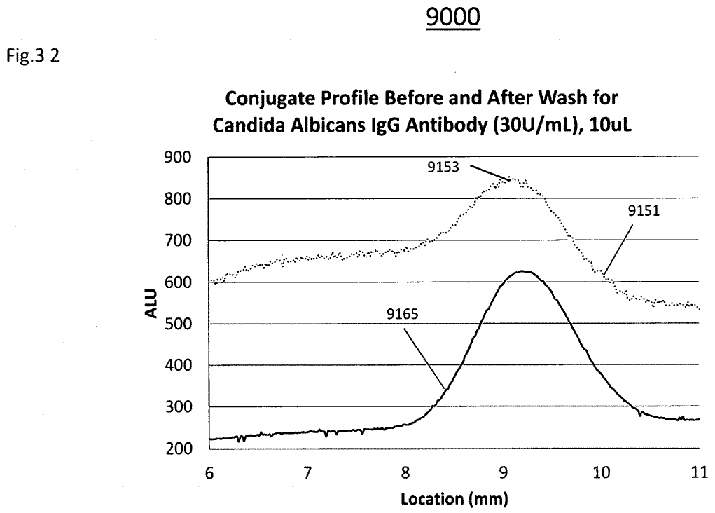

FIG. 32 shows the conjugate concentration profiles in ALU during incubation 9151 (both free and bound conjugate) and after wash 9165 (with bound conjugate at the capture zone) for the lateral flow assay Candida albicans IgG Antibody. In this experiment, 10 μL of sample fluid (Ab concentration 30U/mL) is added to the sample zone for it to flow, dissolve, and move the conjugate to the capture zone for a 5-minute incubation in ambient conditions. After incubation, a 20 uL wash fluid is added to the sample pad, removing the unbound conjugate. 9153 is the peak of the conjugate during incubation.

### PARTS LIST FOR FIGS. 1-32

100 immunoassay (lateral flow) device
104 assay chip
105 base or support layer, chip
106 porous layer, chip
108 solid frame
110 top cover
112 bottom cover
116 fluid metering window
120 air vent
124 scan window
130 sample pad
134 sample (first) fluid
135 wet-dry interface
136 flow direction
138 second (wash) fluid
140 sample zone
150 conjugate zone
151 dissolved conjugate distribution profile
151A wider dissolved conjugate distribution
151B narrower dissolved conjugate distribution
153 peak conjugate concentration location
153A peak conjugate concentration location
153B peak conjugate concentration location
153C peak conjugate concentration location
154 conjugate
155 bound conjugate signal in read area
155A bound conjugate signal in read area
155B bound conjugate signal in read area
155C bound conjugate signal in read area
156 porous layer, smaller pore size
157 porous layer, larger pore size
160 capture zone
160A capture zone
160B capture zone
160C capture zone
161 read area
162 capture antibodies, beads
165 bound conjugate concentration signal, post wash
165A bound conjugate concentration signal
165B bound conjugate concentration signal
166 scanned conjugate signal following wash
166A scanned conjugate signal
166B scanned conjugate signal
166C scanned conjugate signal
170 waste (absorption) zone
174 waste (absorption) pad
176 scanning instrument (optical sensor)
178 scan direction
180 spacer structure
184 nubs
190 air gap
194 excitation wavelength
196 emission wavelength
300 lateral flow device
3000 lateral flow device
3008 frame
3110 top cover
3112 bottom cover
3116 sample metering window
3124 scanning window
3130 sample pad
3140 sample area or zone
3150 conjugate area or zone
3160 capture zone
3170 waste (absorption) zone
3174 absorption pad
3180 spacer structure
3184 nubs
4000 lateral flow device
4104 chip
4108 frame
4110 top cover
4112 bottom cover
4116 sample metering window
4124 scanning window
4130 sample pad
4140 sample zone
4150 conjugate zone
4160 capture zone
4170 waste zone
4174 absorption zone
4180 spacer structure
4184 nubs
5000 lateral flow device
5104 chip
5108 frame
5110 top cover
5112 bottom cover
5116 sample metering window
5124 scanning window
5130 sample pad
5140 sample zone
5150 conjugate zone
5160 capture zone
5170 waste zone
5174 absorption pad
5180 spacer structure
5184 nubs
6000 lateral flow device
6008 frame
6110 top cover
6112 bottom cover
6116 sample metering window
6124 scanning window
6130 sample pad
6140 sample area
6150 conjugate area or zone
6160 capture zone
6170 waste (absorption) zone
6174 absorption pad
6180 spacer structure
6184 nubs
7000 lateral flow device
7008 frame
7110 top cover
7112 bottom cover
7116 sample metering window
7124 scanning window
7130 sample pad
7140 sample area
7150 conjugate area or zone
7160 capture zone
7170 waste (absorption) zone
7174 absorption pad
7180 spacer structure
7184 nubs
8000 lateral flow device
8008 frame
8110 top cover
8112 bottom cover
8116 sample metering window
8124 scanning windows
9151 profile, conjugate concentration signal
9153 peak, conjugate concentration signal
9165 profile, conjugate concentration signal

It will be understood that other variations and modifications can be made to the embodiments described herein in the spirit and scope of the invention, and as defined by the following claims.

1. An immunoassay device comprising:
a frame defining an interior;
an assay chip disposed within the interior;
a sample zone including a sample pad adjacent one end of the assay chip, in which the sample zone that is configured to receive a first sample fluid having at least one analyte of interest, the assay chip being made from a porous hydrophilic material that enables fluid transport;
at least one conjugate zone downstream of the sample zone, the conjugate zone comprising a conjugate material;
at least one capture zone downstream of the at least one conjugate zone having at least one capture antibody; and
at least one waste zone downstream of the at least one capture zone, wherein the assay chip comprises a transparent support layer and a porous layer capable of fluid transport.

2. The device according to claim 1, wherein the assay chip comprises a plurality of capture zones disposed downstream of the conjugate zone.

3. The device according to claim 2, in which the capture zones are disposed serially on the assay chip.

4. The device according to claim 1, wherein the waste zone comprises an absorption pad made from a porous material attached to the end of the assay chip opposite the end forming the sample zone.

5. The device according to claim 1, comprising a plurality of assay chips, each of the assay chips comprising at least one conjugate zone, at least one capture zone and having opposing ends forming part of a sample zone and a waste zone.

6. The device according to claim 5, in which each of the plurality of assay chips are disposed in parallel spaced relation within the interior of the device.

7. The device according to claim 6, further comprising a plurality of sample pads, each sample pad being attached to one end of a said assay chip at the sample zone, the device having a plurality of sample zones and waste zones.

8. The device according to claim 1, further comprising a spacer structure disposed within the interior of the device, the spacer structure including at least one feature for maintaining the assay chip in a position and minimize chances for fluid wicking flow between the chip and the frame.

9. The device according to claim 1, further comprising a solid frame including a top cover and a bottom cover.

10. The device according to claim 9, wherein the top cover includes a formed fluid metering window disposed and aligned with the sample pad.

11. The device according to claim 9, further comprising a scan window disposed in the frame, the scan window extending longitudinally and aligned with the conjugate zone, the capture zone and at least a portion of the waste zone.

12. The device according to claim 11, in which the scan window is formed in one of the top cover or the bottom cover.

13. The device according to claim 10, in which the assay chip comprises an optically transparent base layer and a porous layer.

14. The device according to claim 13, in which the porous layer is defined by a first layer and a second layer, each layer having different sized pore diameters.

15. The device according to claim 14, further comprising a plurality of beads disposed in the conjugate layer, wherein the first and second layers of the porous layer of the chip creates a flow velocity gradient, and further creates a wider distribution of dissolved conjugate into the capture zone.

16. The device according to claim 15, wherein the at least one capture layer includes hydrophilic beads having coated antibodies.

17. The device according to claim 1, wherein the sample pad extends above at least a portion of the conjugate zone.

18. A method of performing an assay using a lateral flow device, the device comprising a sample zone, a conjugate zone downstream of the sample zone containing a labeled conjugate, at least one capture zone downstream of the conjugate zone having a capture antibody, and a waste zone downstream of the at least one capture zone, in which the sample zone includes a sample pad and the waste zone includes an absorption pad and in which the sample pad and absorption pads are coupled to opposing ends of a chip containing the conjugate and capture zones, the method comprising the steps of:
dispensing a sample fluid having at least one analyte of interest onto the sample pad in the sample zone in which the dispensed sample flows along the chip from the sample zone and dissolves labeled conjugate in the conjugate zone, moving the dissolved conjugate in solution to the capture zone, in which volume of the sample fluid is controlled to stop bulk fluid flow while at least a portion of the waste zone is still dry;
incubating the solution with conjugate, analyte and capture antibody for an assay specific time period;
following incubation, dispensing a second fluid to the sample pad to resume fluid flow and in which unbound conjugate is removed from the at least one capture zone with fluid flowing toward the waste zone; and
optically scanning the bound conjugate in the capture zone.

19. The method according to claim 18, in which the optical scanning comprises taking a first optical scan and a second optical scan along a scan window of the device in which the first optical scan is taken immediately before the application of the second fluid and the second optical scan is taken after the second fluid has been dispensed wherein each optical scan obtains a conjugate concentration signal as a function of time and location on the device.

20. The method according to claim 19, wherein the first optical scan obtains a total conjugate concentration signal profile and the second optical scan obtains a bound conjugate concentration signal profile.

21. The method according to claim 20, further comprising determining the location of a peak signal of the total conjugate concentration profile as a read area.

22. The method according to claim 21, further comprising optically scanning the device to determine the edges of the capture zone and verifying that the read area is within the capture zone.

23. The method according to claim 21, further comprising obtaining a normalization value of the total conjugate signal profile in the read area prior to dispensing the second fluid.

24. The method according to claim 23, further comprising scaling the scanned conjugate signal in the read area during the second optical scan based on the obtained normalization value.

25. The method according to claim 18, in which the device includes a single chip containing a plurality of capture zones configured to detect different analytes of interest.

26. The method according to claim 18, in which the device includes a plurality of chips arranged in spaced relation, each chip having a conjugate zone and at least one capture zone.

27. The method according to claim 18, further comprising establishing a flow velocity gradient in the chip in at least the conjugate zone.

28. The method according to claim 27, including providing the chip with two porous layers, a first porous layer and a second porous layer in which the first porous layer has pores with larger diameters than the pores of the second porous layer.

29. The method according to claim 28, including providing beads with coated antibodies in the capture zone.

30. The method according to claim 27, wherein the sample pad is disposed above at least a portion of the conjugate layer.
